(19) United States
(12) Patent Application Publication
(10) Pub. No.: US 2021/0002209 A1
(43) Pub. Date: Jan. 7, 2021

(54) NOVEL CRYSTALLINE FORMS OF TAMIBAROTENE FOR TREATMENT OF CANCER

(71) Applicant: TRANSGENEX NANOBIOTECH, INC., Tampa, FL (US)

(72) Inventors: Mazen HANNA, Tampa, FL (US); Manomi PERERA, Tampa, FL (US); Jiyu YAN, Tampa, FL (US); Andrew HANNA, Lutz, FL (US)

(21) Appl. No.: 16/969,261

(22) PCT Filed: Feb. 11, 2019
(86) PCT No.: PCT/US2019/017480

§ 371 Date: Aug. 12, 2020

Related U.S. Application Data
(60) Provisional application No. 62/629,892, filed on Feb. 13, 2018.

(51) Int. Cl. C07C 233/66; C07C 53/126; C07C 55/12; C07C 55/14; C07C 57/145; C07C 57/15; C07C 57/30; C07C 59/265; C07C 65/03; C07C 63/331

(52) U.S. Cl. CPC C07C 233/66; C07C 53/126; C07C 55/12; C07C 55/14; C07C 57/145; C07B 2200/13; C07C 57/30; C07C 59/265; C07C 65/03; C07C 63/331; C07C 57/15

(57) ABSTRACT

Synthesis and characterization of novel tamibarotene forms suitable for pharmaceutical compositions in drug delivery systems to treat human or warm-blooded mammal diseases.

### CROSS-REFERENCE TO RELATED APPLICATIONS

This application claims priority to U.S. Provisional Application No. 62/629,892, filed Feb. 13, 2018, which is incorporated herein by reference.

### FIELD OF THE INVENTION

This disclosure pertains to novel crystalline forms of tamibarotene and pharmaceutical compositions comprising the same. The tamibarotene compositions can be used for the safe and effective treatment of human or warm-blooded mammal diseases including a variety of cancers, including drug resistant and radio-resistant cancers, Alzheimer's disease, Crohn's disease, autoimmune diseases, rheumatoid arthritis, and non-alcoholic fatty liver disease. The novel forms include but are not limited to cocrystals, salts, solvates of salts, and mixtures thereof. Methods for the preparation of and pharmaceutical compositions suitable for drug delivery systems that include one or more of these new forms are also disclosed.

### BACKGROUND OF THE INVENTION

Tamibarotene, a synthetic retinoid first reported in 2007 [Miwako et al. (2007) Drugs Today (Barc) 43(8):563-68], is a white crystalline powder with the empirical formula of C₂₂H₂₅NO₃ and the IUPAC name as 4-[(5,5,8,8-tetramethyl-5,6,7,8-tetrahydronaphthalen-2-yl)carbamoyl]benzoic acid. Tamibarotene is considered as a stable derivative of retinoic acid and the structural formula (I) with mainly two rigid benzene rings joined by an amide bond. Tamibarotene is soluble in DMF, methanol, ethanol, DMSO, and other organic solvents. However, tamibarotene is insoluble in acetonitrile, water, and various other buffer solutions (pH 3^{˜}7) (Patent Application No. CN101200435 (B)).

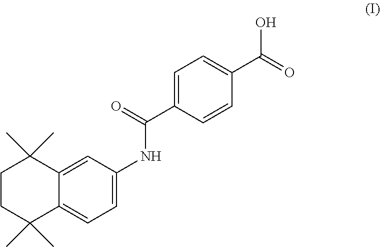 [SMILES: CC1(C)CCC(C)(C)c2cc(NC(=O)c3ccc(C(=O)O)cc3)ccc21]

Tamibarotene is a specific agonist for retinoic acid receptor alpha/beta with possible binding to retinoid X receptors (RXR)^{Drug bank}. This drug is also called retinobenzoic acid and approved for treatment of relapsed and refractory acute promyelocytic leukemia in Japan in 2005 under the brand name Amnolake® [Miwako et al. (2007) Drugs Today (Barc) 43(8):563-68; “Tamibarotene: AM 80, retinobenzoic acid, Tamibaro” (2004) Drugs in R&D 5(6):359-62].

Tamibarotene was developed to overcome all trans retinoic acid (ATAR) resistance and early trials has shown it has a better tolerant than ATAR and also has shown potential antineoplastic activity. Also, tamibarotene is in clinical trials against acute promyelocytic leukemia and may be used in other cancers including liver cancer and solid tumors. Cancers continue to constitute a major cause of morbidity and mortality worldwide. Traditional chemotherapies often cannot completely eradicate tumors, prevent cancer recurrence, or prevent metastasis in lung cancer patients. Recently, in some cases, these failures in effectively treating cancers have been attributed to cancer stem cells (CSCs), which have properties of self-renewal, tumor initiation, and tumor maintenance, and are considered a major cause of mortality after relapse following treatment. CSCs manage to escape chemotherapies and seed new tumor growth, due to the survival of quiescent CSCs [Clarke et al. (2006) Cancer Res. 66:9339-44; Reya et al. (2001) Nature 414:105-11]. With growing evidence supporting the role of CSCs in tumorigenesis [Gupta et al. (2009) Nat. Med. 15:1010-12], tumor heterogeneity [Meacham et al. (2013) Nature 501:328-37], resistance to chemotherapeutic and radiation therapies [Li et al. (2008) J. Natl. Cancer Inst. 100:672-9; Diehn et al. (2009) Nature 458:780-3], and the metastatic phenotype [Shiozawa et al. (2013) Pharmacol. Ther. 138:285-93], the development of specific therapies that target CSCs holds promise for improving the survival and quality of life for cancer patients, especially those with metastatic disease [Takebe et al. (2011) Nat. Rev. Clin. Oncol. 8:97-106; Dalerba et al. (2007) Cell Stem Cell 1:241-2]. Thus, there is a continuing and urgent need for the development of novel therapeutic agents that target CSCs.

Tamibarotene is also being investigated in possible treatment for Alzheimer's disease, multiple myeloma, Crohn's disease [Fukasawa et al. (2012) Biological & Pharmaceutical Bulletin 35(8):1206-12], and chronic obstructive pulmonary disease [Sakai et al. (2014) J Control Release 196:154-60].

There is very little information available on manipulation of the solid forms of tamibarotene. Crystal structure of tamibarotene is published in the Cambridge Structural Database [CSD February 2017 update] [Toriumi et al. (1990) J. Org. Chem. 55:259]. However, there are at least two reported crystal polymorphs of tamibarotene, type 1 and type II, which have different melting points. Type I crystals melts at 193° C. and type 2 crystals melts at 233° C. Type 1 crystals are considered extremely difficult to synthesize as there can be transitions between crystal forms with a physical impact. Hence, type 1 crystals are considered unsuitable as a raw material for mass preparation of a pharmaceutical product, which has a uniform standard. Type 2 crystals have higher stability not only for a physical impact, but also for heat, temperature and light and has more advantage in pharmaceutical industry (U.S. Pat. No. 8,252,837 B2).

Tamibarotene is available as a tablet for oral suspension, which contain 2 mg of free tamibarotene and the recommended dose is 6 mg/m² in two divided doses (www.Pharmacodia.com (2012). Tamibarotene has shown favorable pharmacokinetic profile and milder side effects than ATRA in clinical trials [Miwako et al. (2007) Drugs Today (Barc) 43(8):563-68]. There are additional clinical trials underway in evaluating the efficacy of tamibarotene in maintenance therapy of APL (Acute promyelocytic leukemia) and other diseases like tumors and autoimmune diseases. Since tamibarotene is, relatively, a new drug there is a larger area to study the effectiveness of tamibarotene, which would be beneficial for the development of the pharmaceutical industry. Tamibarotene has a very poor water solubility (0.000575 mg/mL)(^{drug bank}). Hence, it is beneficial in investigating new solid forms of tamibarotene with improved solubility and bioavailability.

There is very little information available in the Cambridge Structural Database (CSD February 2017 update) on attempts, prior to this invention, towards designing molecular complex of tamibarotene (tamibarotene and a cocrystal former) that would be beneficial in enhancing the physicochemical properties of the parent drug and co-former derived from. Such properties include melting point, thermal and electrical conductivity, aqueous solubility rate of dissolution, permeability, and potentially its clinical profile. It is for the first time that the concept of a molecular complex by design to assist improving the physicochemical properties of tamibarotene has been discussed here.

### SUMMARY OF THE INVENTION

The present disclosure is directed towards generating new forms of tamibarotene that have improved physicochemical characteristics. One aspect of the present disclosure includes novel molecular complexes of tamibarotene neutral and ionic that includes cocrystals, salts, and solvates (e.g., hydrates and mixed solvates as well as solvates of salts), and mixtures containing such materials. In addition, the disclosure further includes methods for the preparation of such complexes.

The disclosure further includes compositions of molecular complexes of tamibarotene suitable for incorporation in a pharmaceutical dosage form. Specific molecular complexes pertaining to the disclosure include, but are not limited to, complexes of tamibarotene and adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, and L-malic acid. Obvious variants of the disclosed tamibarotene forms in the text, including those described by the drawings and examples, will be readily apparent to the person of ordinary skill in the art having the present disclosure, and such variants are considered to be a part of the current invention.

The disclosure also includes results of characterization of the new molecular complexes by PXRD and FTIR confirming their novelty compared with that of their parent molecule and the conformer.

The foregoing and other features and advantages of the disclosed technology will become more apparent from the following detailed description, which proceeds with reference to the accompanying drawings. Such description is meant to be illustrative, but not limiting, of the invention.

### BRIEF DESCRIPTION OF THE DRAWINGS

FIG. 1. PXRD profile of novel tamibarotene:adipic acid form. (middle profile).

FIG. 2. FTIR spectrum of tamibarotene:adipic acid novel form. (middle spectrum).

FIG. 3. PXRD profile of novel tam ibarotene:DL-aspartic acid form. (middle profile).

FIG. 4. FTIR spectrum of tamibarotene:DL-aspartic acid novel form. (middle spectrum).

FIG. 5. PXRD profile of novel form of tamibarotene:acetylsalicylic acid. (middle profile).

FIG. 6. FTIR spectrum of tamibarotene:acetylsalicylic acid novel form. (middle spectrum).

FIG. 7. PXRD profile of novel form tamibarotene:biphenyl-4-carboxylic acid. (middle profile).

FIG. 8. FTIR spectrum of novel form tamibarotene:biphenyl-4-carboxylic acid. (middle spectrum).

FIG. 9. PXRD profile of novel form tamibarotene:caffeic acid. (middle profile).

FIG. 10. FTIR spectrum of novel tamibarotene:caffeic acid. (middle spectrum).

FIG. 11. PXRD profile of novel form tamibarotene:decanoic acid. (middle profile).

FIG. 12. FTIR spectrum of novel tamibarotene:decanoic acid. (middle spectrum).

FIG. 13. PXRD profile of novel form tamibarotene:diphenic acid. (middle profile).

FIG. 14. FTIR spectrum of novel tamibarotene:diphenic acid. (middle spectrum).

FIG. 15. PXRD profile of novel form tamibarotene:gallic acid. (middle profile).

FIG. 16. FTIR spectrum of novel tamibarotene:gallic acid. (middle spectrum).

FIG. 17. PXRD profile of novel form tamibarotene:fumaric acid. (middle profile).

FIG. 18. FTIR spectrum of novel tamibarotene:fumaric acid. (middle spectrum).

FIG. 19. PXRD profile of novel form tamibarotene:ibuprofen. (middle profile).

FIG. 20. FTIR spectrum of novel tamibarotene:ibuprofen. (middle spectrum).

FIG. 21. PXRD profile of novel form tamibarotene:maleic acid. (Top profile 5C is the tamibarotene:maleic acid new form. 5P is tamibarotene after evaporating in an acetone slurry. Tami batch 1 is the starting material.).

FIG. 22. FTIR spectrum of novel tamibarotene:maleic acid. (Tami batch 2 is the starting material. TGX-2-5C is tamibarotene:maleic acid new form.).

FIG. 23. PXRD profile of novel form tamibarotene:nicotinamide (Top profile 4F is type II of anhydrous tamibarotene. 5E is tamibarotene:nicotinamide novel form. 5P is tamibarotene after evaporating in an acetone slurry. Tami batch 1 is the starting material.).

FIG. 24. FTIR spectrum of novel tamibarotene:nicotinamide. (Top spectrum Tami batch 1 is the starting material. TGX-2-5E is tamibarotene:nicotinamide novel form.).

FIG. 25. PXRD profile of novel form tamibarotene:isonicotinamide (Top profile 4F is type II of anhydrous tamibarotene. 5F is novel tamibarotene:isonicotinamide form. 5P is tamibarotene after evaporating in an acetone slurry. Tami batch 1 is the starting material.).

FIG. 26. FTIR spectrum of novel tamibarotene:isonicotinamide. (Top spectrum Tami is the starting material. TGX-2-5F is the novel tamibarotene:isonicotinamide.).

FIG. 27. PXRD profile of novel form tamibarotene:citric acid. (Top profile 5G is novel tamibarotene:citric acid. 5P is tamibarotene after evaporating in an acetone slurry. Tami batch 1 is the starting material.).

FIG. 28. FTIR spectrum of novel tam ibarotene:citric acid. (Tami batch 2 is the starting material. Middle spectrum is citric acid and TGX-2-5G is novel tamibarotene:citric acid.).

FIG. 29. PXRD profile of novel form tamibarotene:nicotinic acid. (5N is novel tamibarotene:nicotinic acid. Tami form II is anhydrous type II of tamibarotene. 5P is tamibarotene after evaporating in an acetone slurry. Tami batch 1 is the starting material.).

FIG. 30. FTIR spectrum of novel tamibarotene:nicotinic acid. (Tami batch 2 is the starting material. TGX-2-5N is the novel tamibarotene:nicotinic acid.).

FIG. 31. PXRD profile of novel form tamibarotene:3,4-dihydroxybenzoic acid. (7E is the novel tamibarotene:3,4-dihydroxybenzoic acid. 4F is anhydrous type II of tamibarotene. 5P is tamibarotene after evaporating in an acetone slurry. Tamibarotene form 1 is the starting material.).

FIG. 32. FTIR spectrum of novel tamibarotene:3,4-dihydroxybenzoic acid. (Tami (abbreviation for tamibarotene) is the starting material. TGX-2-7E is the novel tamibarotene:3,4-dihydroxybenzoic acid.).

FIG. 33. PXRD profile of novel form tamibarotene:glutaric acid. (5A is novel tamibarotene:glutaric acid generated form an acetone slurry and 4A is novel tamibarotene:glutaric acid generated form an acetonitrile slurry. 5P is tamibarotene after evaporating in an acetone slurry. Tami (abbreviation for tamibarotene) batch 1 is the starting material.).

FIG. 34. FTIR spectrum of novel tamibarotene:glutaric acid. (Tami (abbreviation for tamibarotene) batch 2 is the starting material. TGX-2-5A is the novel tamibarotene:glutaric acid form.).

FIG. 35. PXRD profile of novel form tamibarotene:L-malic acid. (middle spectrum).

FIG. 36. FTIR spectrum of novel tamibarotene:L-malic acid. (bottom spectrum).

FIG. 37. PXRD profile of tamibarotene:biphenyl-4-carboxylic acid product, 10× scaled up.

FIG. 38. PXRD profile of tamibarotene:diphenic acid product, 10× scaled up.

FIG. 39. PXRD profile of tamibarotene:gallic acid product, 10× scaled up.

FIG. 40. PXRD profile of tamibarotene:ibuprofen product, 10× scaled up.

FIG. 41. PXRD profile of tamibarotene:nicotinamide product, 10× scaled up.

FIG. 42. PXRD profile of tamibarotene:glutaric acid product, 10× scaled up.

FIG. 43. PXRD profile of tamibarotene:biphenyl-4-carboxylic acid after one year of stability testing.

FIG. 44. PXRD profile of tamibarotene:diphenic acid after one year of stability testing.

FIG. 45. PXRD profile of tamibarotene:gallic acid after one year of stability testing.

FIG. 46. PXRD profile of tamibarotene:ibuprofen after one year of stability testing.

FIG. 47. PXRD profile of tamibarotene:nicotinamide after one year of stability testing.

FIG. 48. PXRD profile of tamibarotene:glutaric acid after one year of stability testing.

FIG. 49. Sphere image area of graph sarcoma cells treated with tamibarotene:gallic acid molecular complex.

FIG. 50. Images of sarcoma cell spheres at different concentration of tamibarotene:gallic acid molecular complex.

### DETAILED DESCRIPTION OF THE INVENTION

Active pharmaceutical ingredients (APIs) in pharmaceutical compositions can be prepared in a variety of different chemical forms including: chemical derivatives, solvates, hydrates, cocrystals, and/or salts. Such compounds can also be prepared to have different physical forms. For example, they may be amorphous, may have different crystalline polymorphs, or may exist in different solvated or hydrated states. The discovery of new forms of a pharmaceutically useful compound may provide an opportunity to improve the performance characteristics of a pharmaceutical product. Additionally, it expands the array of resources available for designing, for example, a pharmaceutical dosage form of a drug with a targeted release profile or other desired characteristics.

A specific characteristic that can be targeted includes the crystal form of an API. By altering the crystal form, it therefore becomes possible to vary the physical properties of the target molecule. For example, crystalline polymorphs typically have different aqueous solubility from one another, such that a more thermodynamically stable polymorph is less soluble than a less thermodynamically stable polymorph. In addition to water solubility, pharmaceutical polymorphs can also differ in properties such as rate of dissolution, shelf life, bioavailability, morphology, vapor pressure, density, color, and compressibility. Accordingly, it is desirable to enhance the properties of an active pharmaceutical compound by forming molecular complexes such as a cocrystal, a salt, a solvate or hydrate with respect to aqueous solubility, rate of dissolution, bioavailability, Cmax, Tmax, physicochemical stability, down-stream processibility (e.g., flowability compressibility, degree of brittleness, particle size manipulation), crystallization of amorphous compounds, decrease in polymorphic form diversity, toxicity, taste, production costs, and manufacturing methods.

During the development of drugs in an oral delivery setting, it is frequently advantageous to have novel crystalline forms of such drug materials that possess improved properties, including increased aqueous solubility and stability. It is also desirable, in general, to increase the dissolution rate of such solid forms and potentially increase their bioavailability. This also applies to the development of novel forms of tamibarotene which, when administered orally to a subject could achieve a greater or similar bioavailability and PK profile when compared to an IV or other formulations on a dose-for-dose basis.

Cocrystals, salts, solvates, and hydrates of tamibarotene of the present invention could give rise to improved properties. For example, a new tamibarotene form is particularly advantageous if it can improve the oral bioavailability or the clinical profile of the IV version by cutting the dose for instance. A number of novel tamibarotene forms have been synthesized, characterized, and disclosed herein.

The techniques and approaches set forth in the present disclosure can further be used by the person of ordinary skill in the art to prepare variants thereof, said variants are considered to be part of the inventive disclosure.

The present invention further includes compositions of molecular complexes of tamibarotene suitable for incorporation in a pharmaceutical dosage form. Specific molecular complexes pertaining to the disclosure include, but are not limited to, complexes of tamibarotene and adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, and L-malic acid, which are capable of complexing through solvent evaporation of their solution in single or mixed solvent systems, and slurry suspension.

In one aspect, the invention provides for a molecular complex of tamibarotene and a former selected from the group consisting of: adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, and L-malic acid. In one embodiment, the molecular complex is a crystalline form of tamibarotene and a former selected from the group consisting of: adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, and L-malic acid. In one embodiment, the crystalline form is a cocrystal of tamibarotene and a cocrystal former selected from the group consisting of: adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, and L-malic acid. Crystalline forms between tamibarotene and a former, e.g., cocrystal former, are denoted using a “:” between tamibarotene and the name of the former, i.e., tamibarotene:“former”.

In one embodiment, the crystalline form is a tamibarotene:adipic acid crystalline form. In another embodiment, the crystalline form of tamibarotene:adipic acid is a 1:1 complex. In another embodiment, the tamibarotene:adipic acid crystalline form is a co-crystal. In another embodiment, the tamibarotene:adipic acid crystalline form is characterized by a powder X-ray diffraction pattern comprising a powder X-ray diffraction peak selected from about 10.5, 12.0, 14.5, 22.0, or 26.0° 2θ±0.2° 2θ. In another embodiment, the tamibarotene:adipic acid crystalline form is characterized by a powder X-ray diffraction pattern comprising a powder X-ray diffraction peak at about 10.5° 2θ±0.2° 2θ. In another embodiment, the tamibarotene:adipic acid crystalline form is characterized by a powder X-ray diffraction pattern comprising a powder X-ray diffraction peak at about 12.0° 2θ±0.2° 2θ. In another embodiment, the tamibarotene:adipic acid crystalline form is characterized by a powder X-ray diffraction pattern comprising a powder X-ray diffraction peak at about 14.5° 2θ±0.2° 2θ. In another embodiment, the tamibarotene:adipic acid crystalline form is characterized by a powder X-ray diffraction pattern comprising a powder X-ray diffraction peak at about 22.0° 2θ±0.2° 2θ. In another embodiment, the tamibarotene:adipic acid crystalline form is characterized by a powder X-ray diffraction pattern comprising a powder X-ray diffraction peak at about 26.0° 2θ±0.2° 2θ. In another embodiment, the tamibarotene:adipic acid crystalline form is characterized by a powder X-ray diffraction pattern comprising any four powder X-ray diffraction peaks selected from about 10.5, 12.0, 14.5, 22.0, or 26.0° 2θ±0.2° 2θ. In another embodiment, the tamibarotene:adipic acid crystalline form is characterized by a powder X-ray diffraction pattern comprising any three powder X-ray diffraction peaks selected from about 10.5, 12.0, 14.5, 22.0, or 26.0° 2θ±0.2° 2θ. In another embodiment, the tamibarotene:adipic acid crystalline form is characterized by a powder X-ray diffraction pattern comprising any two powder X-ray diffraction peaks selected from about 10.5, 12.0, 14.5, 22.0, or 26.0° 2θ±0.2° 2θ. In another embodiment, the tamibarotene:adipic acid crystalline form is characterized by a powder X-ray diffraction pattern comprising powder X-ray diffraction peaks at about 10.5, 12.0, 14.5, 22.0, and 26.0° 2θ±0.2° 2θ.

In another embodiment, the crystalline form is a tamibarotene:DL-aspartic acid crystalline form. In one embodiment, the crystalline form of tamibarotene:DL-aspartic acid is a 1:1 complex. In another embodiment, the tamibarotene:DL-aspartic acid crystalline form is a co-crystal. In one embodiment, the tamibarotene:DL-aspartic acid crystalline form is characterized by a powder X-ray diffraction pattern comprising a powder X-ray diffraction peak selected from about 6.5, 10.0, 11.5, or 19.5° 2θ±0.2° 2θ. In another embodiment, the tamibarotene:DL-aspartic acid crystalline form is characterized by a powder X-ray diffraction pattern comprising a powder X-ray diffraction peak at about 6.5° 2θ±0.2° 2θ. In another embodiment, the tamibarotene:DL-aspartic acid crystalline form is characterized by a powder X-ray diffraction pattern comprising a powder X-ray diffraction peak at about 10.0° 2θ±0.2° 2θ. In another embodiment, the tamibarotene:DL-aspartic acid crystalline form is characterized by a powder X-ray diffraction pattern comprising a powder X-ray diffraction peak at about 11.5° 2θ±0.2° 2θ. In another embodiment, the tamibarotene:DL-aspartic acid crystalline form is characterized by a powder X-ray diffraction pattern comprising a powder X-ray diffraction peak at about 19.5° 2θ±0.2° 2θ. In another embodiment, the tamibarotene:DL-aspartic acid crystalline form is characterized by a powder X-ray diffraction pattern comprising any three powder X-ray diffraction peaks selected from about 6.5, 10.0, 11.5, or 19.5° 2θ±0.2° 2θ. In another embodiment, the tamibarotene:DL-aspartic acid crystalline form is characterized by a powder X-ray diffraction pattern comprising any two powder X-ray diffraction peaks selected from about 6.5, 10.0, 11.5, or 19.5° 2θ±0.2° 2θ. In another embodiment, the tamibarotene:DL-aspartic acid crystalline form is characterized by a powder X-ray diffraction pattern comprising powder X-ray diffraction peaks at about 6.5, 10.0, 11.5, and 19.5° 2θ±0.2° 2θ.

In another embodiment, the crystalline form is a tamibarotene:acetylsalicylic acid crystalline form. In one embodiment, the crystalline form of tamibarotene:acetylsalicylic acid is a 1:1 complex. In another embodiment, the tamibarotene:acetylsalicylic acid crystalline form is a co-crystal. In one embodiment, the tamibarotene:acetylsalicylic acid crystalline form is characterized by a powder X-ray diffraction pattern comprising a powder X-ray diffraction peak selected from about 8.0, 8.5, 15.5, 23.0, or 27.0° 2θ±0.2° 2θ. In another embodiment, the tamibarotene:acetylsalicylic acid crystalline form is characterized by a powder X-ray diffraction pattern comprising a powder X-ray diffraction peak at about 8.0° 2θ±0.2° 2θ. In another embodiment, the tamibarotene:acetylsalicylic acid crystalline form is characterized by a powder X-ray diffraction pattern comprising a powder X-ray diffraction peak at about 8.5° 2θ±0.2° 2θ. In another embodiment, the tamibarotene:acetylsalicylic acid crystalline form is characterized by a powder X-ray diffraction pattern comprising a powder X-ray diffraction peak at about 15.5° 2θ±0.2° 2θ. In another embodiment, the tamibarotene:acetylsalicylic acid crystalline form is characterized by a powder X-ray diffraction pattern comprising a powder X-ray diffraction peak at about 23.0° 2θ±0.2° 2θ. In another embodiment, the tamibarotene:acetylsalicylic acid crystalline form is characterized by a powder X-ray diffraction pattern comprising a powder X-ray diffraction peak at about 27.0° 2θ±0.2° 2θ. In another embodiment, the tamibarotene:acetylsalicylic acid crystalline form is characterized by a powder X-ray diffraction pattern comprising any four powder X-ray diffraction peaks selected from about 8.0, 8.5, 15.5, 23.0, or 27.0° 2θ±0.2° 2θ. In another embodiment, the tamibarotene:acetylsalicylic acid crystalline form is characterized by a powder X-ray diffraction pattern comprising any three powder X-ray diffraction peaks selected from about 8.0, 8.5, 15.5, 23.0, or 27.0° 2θ±0.2° 2θ. In another embodiment, the tamibarotene:acetylsalicylic acid crystalline form is characterized by a powder X-ray diffraction pattern comprising any two powder X-ray diffraction peaks selected from about 8.0, 8.5, 15.5, 23.0, or 27.0° 2θ±0.2° 2θ. In another embodiment, the tamibarotene:acetylsalicylic acid crystalline form is characterized by a powder X-ray diffraction pattern comprising powder X-ray diffraction peaks at about 8.0, 8.5, 15.5, 23.0, and 27.0° 2θ±0.2° 2θ.

In another embodiment, the crystalline form is a tamibarotene:biphenyl-4-carboxylic acid crystalline form. In one embodiment, the crystalline form of tamibarotene:biphenyl-4-carboxylic acid is a 1:1 complex. In another embodiment, the tamibarotene:biphenyl-4-carboxylic acid crystalline form is a co-crystal. In one embodiment, the tamibarotene:biphenyl-4-carboxylic acid crystalline form is characterized by a powder X-ray diffraction pattern comprising a powder X-ray diffraction peak selected from about 6.5, 8.0, 8.5, 11.0, 13.0, or 16.0° 2θ±0.2° 2θ. In another embodiment, the tamibarotene:biphenyl-4-carboxylic acid crystalline form is characterized by a powder X-ray diffraction pattern comprising a powder X-ray diffraction peak at about 6.5° 2θ±0.2° 2θ. In another embodiment, the tamibarotene:biphenyl-4-carboxylic acid crystalline form is characterized by a powder X-ray diffraction pattern comprising a powder X-ray diffraction peak at about 8.0° 2θ±0.2° 2θ. In another embodiment, the tamibarotene:biphenyl-4-carboxylic acid crystalline form is characterized by a powder X-ray diffraction pattern comprising a powder X-ray diffraction peak at about 8.5° 2θ±0.2° 2θ. In another embodiment, the tamibarotene:biphenyl-4-carboxylic acid crystalline form is characterized by a powder X-ray diffraction pattern comprising a powder X-ray diffraction peak at about 11.0° 2θ±0.2° 2θ. In another embodiment, the tamibarotene:biphenyl-4-carboxylic acid crystalline form is characterized by a powder X-ray diffraction pattern comprising a powder X-ray diffraction peak at about 13.0° 2θ±0.2° 2θ. In another embodiment, the tamibarotene:biphenyl-4-carboxylic acid crystalline form is characterized by a powder X-ray diffraction pattern comprising a powder X-ray diffraction peak at about 16.0° 2θ±0.2° 2θ. In another embodiment, the tamibarotene:biphenyl-4-carboxylic acid crystalline form is characterized by a powder X-ray diffraction pattern comprising any five powder X-ray diffraction peaks selected from about 6.5, 8.0, 8.5, 11.0, 13.0, or 16.0° 2θ±0.2° 2θ. In another embodiment, the tamibarotene:biphenyl-4-carboxylic acid crystalline form is characterized by a powder X-ray diffraction pattern comprising any four powder X-ray diffraction peaks selected from about 6.5, 8.0, 8.5, 11.0, 13.0, or 16.0° 2θ±0.2° 2θ. In another embodiment, the tamibarotene:biphenyl-4-carboxylic acid crystalline form is characterized by a powder X-ray diffraction pattern comprising any three powder X-ray diffraction peaks selected from about 6.5, 8.0, 8.5, 11.0, 13.0, or 16.0° 2θ±0.2° 2θ. In another embodiment, the tamibarotene:biphenyl-4-carboxylic acid crystalline form is characterized by a powder X-ray diffraction pattern comprising any two powder X-ray diffraction peaks selected from about 6.5, 8.0, 8.5, 11.0, 13.0, or 16.0° 2θ±0.2° 2θ. In another embodiment, the tamibarotene:biphenyl-4-carboxylic acid crystalline form is characterized by a powder X-ray diffraction pattern comprising powder X-ray diffraction peaks at about 6.5, 8.0, 8.5, 11.0, 13.0, and 16.0° 2θ±0.2° 2θ.

In another embodiment, the crystalline form is a tamibarotene:caffeic acid crystalline form. In one embodiment, the crystalline form of tamibarotene:caffeic acid is a 1:1 complex. In another embodiment, the tamibarotene:caffeic acid crystalline form is a co-crystal. In one embodiment, the tamibarotene:caffeic acid crystalline form is characterized by a powder X-ray diffraction pattern comprising a powder X-ray diffraction peak selected from about 3.5, 14.0, 16.0, 17.5, or 27.0° 2θ±0.2° 2θ. In another embodiment, the tamibarotene:caffeic acid crystalline form is characterized by a powder X-ray diffraction pattern comprising a powder X-ray diffraction peak at about 3.5° 2θ±0.2° 2θ. In another embodiment, the tamibarotene:caffeic acid crystalline form is characterized by a powder X-ray diffraction pattern comprising a powder X-ray diffraction peak at about 14.0° 2θ±0.2° 2θ. In another embodiment, the tamibarotene:caffeic acid crystalline form is characterized by a powder X-ray diffraction pattern comprising a powder X-ray diffraction peak at about 16.0° 2θ±0.2° 2θ. In another embodiment, the tamibarotene:caffeic acid crystalline form is characterized by a powder X-ray diffraction pattern comprising a powder X-ray diffraction peak at about 17.5° 2θ±0.2° 2θ. In another embodiment, the tamibarotene:caffeic acid crystalline form is characterized by a powder X-ray diffraction pattern comprising a powder X-ray diffraction peak at about 27.0° 2θ±0.2° 2θ. In another embodiment, the tamibarotene:caffeic acid crystalline form is characterized by a powder X-ray diffraction pattern comprising any four powder X-ray diffraction peaks selected from about 3.5, 14.0, 16.0, 17.5, or 27.0° 2θ±0.2° 2θ. In another embodiment, the tamibarotene:caffeic acid crystalline form is characterized by a powder X-ray diffraction pattern comprising any three powder X-ray diffraction peaks selected from about 3.5, 14.0, 16.0, 17.5, or 27.0° 2θ±0.2° 2θ. In another embodiment, the tamibarotene:caffeic acid crystalline form is characterized by a powder X-ray diffraction pattern comprising any two powder X-ray diffraction peaks selected from about 3.5, 14.0, 16.0, 17.5, or 27.0° 2θ±0.2° 2θ. In another embodiment, the tamibarotene:caffeic acid crystalline form is characterized by a powder X-ray diffraction pattern comprising powder X-ray diffraction peaks at about 3.5, 14.0, 16.0, 17.5, and 27.0° 2θ±0.2° 2θ.

In another embodiment, the crystalline form is a tamibarotene:decanoic acid crystalline form. In one embodiment, the crystalline form of tamibarotene:decanoic acid is a 1:1 complex. In another embodiment, the tamibarotene:decanoic acid crystalline form is a co-crystal. In one embodiment, the tamibarotene:decanoic acid crystalline form is characterized by a powder X-ray diffraction pattern comprising a powder X-ray diffraction peak selected from about 4.0, 14.0, 15.0, 21.5, or 23.5° 2θ±0.2° 2θ. In another embodiment, the tamibarotene:decanoic acid crystalline form is characterized by a powder X-ray diffraction pattern comprising a powder X-ray diffraction peak at about 4.0° 2θ±0.2° 2θ. In another embodiment, the tamibarotene:decanoic acid crystalline form is characterized by a powder X-ray diffraction pattern comprising a powder X-ray diffraction peak at about 14.0° 2θ±0.2° 2θ. In another embodiment, the tamibarotene:decanoic acid crystalline form is characterized by a powder X-ray diffraction pattern comprising a powder X-ray diffraction peak at about 15.0° 2θ±0.2° 2θ. In another embodiment, the tamibarotene:decanoic acid crystalline form is characterized by a powder X-ray diffraction pattern comprising a powder X-ray diffraction peak at about 21.5° 2θ±0.2° 2θ. In another embodiment, the tamibarotene:decanoic acid crystalline form is characterized by a powder X-ray diffraction pattern comprising a powder X-ray diffraction peak at about 23.5° 2θ±0.2° 2θ. In another embodiment, the tamibarotene:decanoic acid crystalline form is characterized by a powder X-ray diffraction pattern comprising any four powder X-ray diffraction peaks selected from about 4.0, 14.0, 15.0, 21.5, or 23.5° 2θ±0.2° 2θ. In another embodiment, the tamibarotene:decanoic acid crystalline form is characterized by a powder X-ray diffraction pattern comprising any three powder X-ray diffraction peaks selected from about 4.0, 14.0, 15.0, 21.5, or 23.5° 2θ±0.2° 2θ. In another embodiment, the tamibarotene:decanoic acid crystalline form is characterized by a powder X-ray diffraction pattern comprising any two powder X-ray diffraction peaks selected from about 4.0, 14.0, 15.0, 21.5, or 23.5° 2θ±0.2° 2θ. In another embodiment, the tamibarotene:decanoic acid crystalline form is characterized by a powder X-ray diffraction pattern comprising powder X-ray diffraction peaks at about 4.0, 14.0, 15.0, 21.5, and 23.5° 2θ±0.2° 2θ.

In another embodiment, the crystalline form is a tamibarotene:diphenic acid crystalline form. In one embodiment, the crystalline form of tamibarotene:diphenic acid is a 1:1 complex. In another embodiment, the tamibarotene:diphenic acid crystalline form is a co-crystal. In one embodiment, the tamibarotene:diphenic acid crystalline form is characterized by a powder X-ray diffraction pattern comprising a powder X-ray diffraction peak selected from about 8.0, 8.5, 13.0, 14.0, 14.5, or 16.0° 2θ±0.2° 2θ. In another embodiment, the tamibarotene:diphenic acid crystalline form is characterized by a powder X-ray diffraction pattern comprising a powder X-ray diffraction peak at about 8.0° 2θ±0.2° 2θ. In another embodiment, the tamibarotene:diphenic acid crystalline form is characterized by a powder X-ray diffraction pattern comprising a powder X-ray diffraction peak at about 8.5° 2θ±0.2° 2θ. In another embodiment, the tamibarotene:diphenic acid crystalline form is characterized by a powder X-ray diffraction pattern comprising a powder X-ray diffraction peak at about 13.0° 2θ±0.2° 2θ. In another embodiment, the tamibarotene:diphenic acid crystalline form is characterized by a powder X-ray diffraction pattern comprising a powder X-ray diffraction peak at about 14.0° 2θ±0.2° 2θ. In another embodiment, the tamibarotene:diphenic acid crystalline form is characterized by a powder X-ray diffraction pattern comprising a powder X-ray diffraction peak at about 14.5° 2θ±0.2° 2θ. In another embodiment, the tamibarotene:diphenic acid crystalline form is characterized by a powder X-ray diffraction pattern comprising a powder X-ray diffraction peak at about 16.0° 2θ±0.2° 2θ. In another embodiment, the tamibarotene:diphenic acid crystalline form is characterized by a powder X-ray diffraction pattern comprising any five powder X-ray diffraction peaks selected from about 8.0, 8.5, 13.0, 14.0, 14.5, or 16.0° 2θ±0.2° 2θ. In another embodiment, the tamibarotene:diphenic acid crystalline form is characterized by a powder X-ray diffraction pattern comprising any four powder X-ray diffraction peaks selected from about 8.0, 8.5, 13.0, 14.0, 14.5, or 16.0° 2θ±0.2° 2θ. In another embodiment, the tamibarotene:diphenic acid crystalline form is characterized by a powder X-ray diffraction pattern comprising any three powder X-ray diffraction peaks selected from about 8.0, 8.5, 13.0, 14.0, 14.5, or 16.0° 2θ±0.2° 2θ. In another embodiment, the tamibarotene:diphenic acid crystalline form is characterized by a powder X-ray diffraction pattern comprising any two powder X-ray diffraction peaks selected from about 8.0, 8.5, 13.0, 14.0, 14.5, or 16.0° 2θ±0.2° 2θ. In another embodiment, the tamibarotene:diphenic acid crystalline form is characterized by a powder X-ray diffraction pattern comprising powder X-ray diffraction peaks at about 8.0, 8.5, 13.0, 14.0, 14.5, and 16.0° 2θ±0.2° 2θ.

In another embodiment, the crystalline form is a tamibarotene:gallic acid crystalline form. In one embodiment, the crystalline form of tamibarotene:gallic acid is a 1:1 complex. In another embodiment, the tamibarotene:gallic acid crystalline form is a co-crystal. In one embodiment, the tamibarotene:gallic acid crystalline form is characterized by a powder X-ray diffraction pattern comprising a powder X-ray diffraction peak selected from about 3.5, 23, 28.5, or 29.5° 2θ±0.2° 2θ. In another embodiment, the tamibarotene:gallic acid crystalline form is characterized by a powder X-ray diffraction pattern comprising a powder X-ray diffraction peak at about 3.5° 2θ±0.2° 2θ. In another embodiment, the tamibarotene:gallic acid crystalline form is characterized by a powder X-ray diffraction pattern comprising a powder X-ray diffraction peak at about 23.0° 2θ±0.2° 2θ. In another embodiment, the tamibarotene:gallic acid crystalline form is characterized by a powder X-ray diffraction pattern comprising a powder X-ray diffraction peak at about 28.5° 2θ±0.2° 2θ. In another embodiment, the tamibarotene:gallic acid crystalline form is characterized by a powder X-ray diffraction pattern comprising a powder X-ray diffraction peak at about 29.5° 2θ±0.2° 2θ. In another embodiment, the tamibarotene:gallic acid crystalline form is characterized by a powder X-ray diffraction pattern comprising any three powder X-ray diffraction peaks selected from about 3.5, 23, 28.5, or 29.5° 2θ±0.2° 2θ. In another embodiment, the tamibarotene:gallic acid crystalline form is characterized by a powder X-ray diffraction pattern comprising any two powder X-ray diffraction peaks selected from about 3.5, 23, 28.5, or 29.5° 2θ±0.2° 2θ. In another embodiment, the tamibarotene:gallic acid crystalline form is characterized by a powder X-ray diffraction pattern comprising powder X-ray diffraction peaks at about 3.5, 23, 28.5, and 29.5° 2θ±0.2° 2θ.

In another embodiment, the crystalline form is a tamibarotene:fumaric acid crystalline form. In one embodiment, the crystalline form of tamibarotene:fumaric acid is a 1:1 complex. In another embodiment, the tamibarotene:fumaric acid crystalline form is a co-crystal. In one embodiment, the tamibarotene:fumaric acid crystalline form is characterized by a powder X-ray diffraction pattern comprising a powder X-ray diffraction peak selected from about 3.0, 6.5, 16.5, 18.0, or 21.5° 2θ±0.2° 2θ. In another embodiment, the tamibarotene:fumaric acid crystalline form is characterized by a powder X-ray diffraction pattern comprising a powder X-ray diffraction peak at about 3.0° 2θ±0.2° 2θ. In another embodiment, the tamibarotene:fumaric acid crystalline form is characterized by a powder X-ray diffraction pattern comprising a powder X-ray diffraction peak at about 6.5° 2θ±0.2° 2θ. In another embodiment, the tamibarotene:fumaric acid crystalline form is characterized by a powder X-ray diffraction pattern comprising a powder X-ray diffraction peak at about 16.5° 2θ±0.2° 2θ. In another embodiment, the tamibarotene:fumaric acid crystalline form is characterized by a powder X-ray diffraction pattern comprising a powder X-ray diffraction peak at about 18.0° 2θ±0.2° 2θ. In another embodiment, the tamibarotene:fumaric acid crystalline form is characterized by a powder X-ray diffraction pattern comprising a powder X-ray diffraction peak at about 21.5° 2θ±0.2° 2θ. In another embodiment, the tamibarotene:fumaric acid crystalline form is characterized by a powder X-ray diffraction pattern comprising any four powder X-ray diffraction peaks selected from about 3.0, 6.5, 16.5, 18.0, or 21.5° 2θ±0.2° 2θ. In another embodiment, the tamibarotene:fumaric acid crystalline form is characterized by a powder X-ray diffraction pattern comprising any three powder X-ray diffraction peaks selected from about 3.0, 6.5, 16.5, 18.0, or 21.5° 2θ±0.2° 2θ. In another embodiment, the tamibarotene:fumaric acid crystalline form is characterized by a powder X-ray diffraction pattern comprising any two powder X-ray diffraction peaks selected from about 3.0, 6.5, 16.5, 18.0, or 21.5° 2θ±0.2° 2θ. In another embodiment, the tamibarotene:fumaric acid crystalline form is characterized by a powder X-ray diffraction pattern comprising powder X-ray diffraction peaks at about 3.0, 6.5, 16.5, 18.0, and 21.5° 2θ±0.2° 2θ.

In one embodiment, the crystalline form is a tamibarotene:ibuprofen crystalline form. In one embodiment, the crystalline form of tamibarotene:ibuprofen is a 1:1 complex. In another embodiment, the tamibarotene:ibuprofen crystalline form is a co-crystal. In one embodiment, the tamibarotene:ibuprofen crystalline form is characterized by a powder X-ray diffraction pattern comprising a powder X-ray diffraction peak selected from about 3.5, 7.0, 17.5, or 19.5° 2θ±0.2° 2θ. In another embodiment, the tamibarotene:ibuprofen crystalline form is characterized by a powder X-ray diffraction pattern comprising a powder X-ray diffraction peak at about 3.5° 2θ±0.2° 2θ. In another embodiment, the tamibarotene:ibuprofen crystalline form is characterized by a powder X-ray diffraction pattern comprising a powder X-ray diffraction peak at about 7.0° 2θ±0.2° 2θ. In another embodiment, the tamibarotene:ibuprofen crystalline form is characterized by a powder X-ray diffraction pattern comprising a powder X-ray diffraction peak at about 17.5° 2θ±0.2° 2θ. In another embodiment, the tamibarotene:ibuprofen crystalline form is characterized by a powder X-ray diffraction pattern comprising a powder X-ray diffraction peak at about 19.5° 2θ±0.2° 2θ. In another embodiment, the tamibarotene:ibuprofen crystalline form is characterized by a powder X-ray diffraction pattern comprising any three powder X-ray diffraction peaks selected from about 3.5, 7.0, 17.5, or 19.5° 2θ±0.2° 2θ. In another embodiment, the tamibarotene:ibuprofen crystalline form is characterized by a powder X-ray diffraction pattern comprising any two powder X-ray diffraction peaks selected from about 3.5, 7.0, 17.5, or 19.5° 2θ±0.2° 2θ. In another embodiment, the tamibarotene:ibuprofen crystalline form is characterized by a powder X-ray diffraction pattern comprising powder X-ray diffraction peaks at about 3.5, 7.0, 17.5, and 19.5° 2θ±0.2° 2θ.

In another embodiment, the crystalline form is a tamibarotene:maleic acid crystalline form. In one embodiment, the crystalline form of tamibarotene:maleic acid is a 1:1 complex. In another embodiment, the tamibarotene:maleic acid crystalline form is a co-crystal. In one embodiment, the tamibarotene:maleic acid crystalline form is characterized by a powder X-ray diffraction pattern comprising a powder X-ray diffraction peak selected from about 4.0, 6.0, 12.5, 14.5, or 17.5° 2θ±0.2° 2θ. In another embodiment, the tamibarotene:maleic acid crystalline form is characterized by a powder X-ray diffraction pattern comprising a powder X-ray diffraction peak at about 4.0° 2θ±0.2° 2θ. In another embodiment, the tamibarotene:maleic acid crystalline form is characterized by a powder X-ray diffraction pattern comprising a powder X-ray diffraction peak at about 6.0° 2θ±0.2° 2θ. In another embodiment, the tamibarotene:maleic acid crystalline form is characterized by a powder X-ray diffraction pattern comprising a powder X-ray diffraction peak at about 12.5° 2θ±0.2° 2θ. In another embodiment, the tamibarotene:maleic acid crystalline form is characterized by a powder X-ray diffraction pattern comprising a powder X-ray diffraction peak at about 14.5° 2θ±0.2° 2θ. In another embodiment, the tamibarotene:maleic acid crystalline form is characterized by a powder X-ray diffraction pattern comprising a powder X-ray diffraction peak at about 17.5° 2θ±0.2° 2θ. In another embodiment, the tamibarotene:maleic acid crystalline form is characterized by a powder X-ray diffraction pattern comprising any four powder X-ray diffraction peaks selected from about 4.0, 6.0, 12.5, 14.5, or 17.5° 2θ±0.2° 2θ. In another embodiment, the tamibarotene:maleic acid crystalline form is characterized by a powder X-ray diffraction pattern comprising any three powder X-ray diffraction peaks selected from about 4.0, 6.0, 12.5, 14.5, or 17.5° 2θ±0.2° 2θ. In another embodiment, the tamibarotene:maleic acid crystalline form is characterized by a powder X-ray diffraction pattern comprising any two powder X-ray diffraction peaks selected from about 4.0, 6.0, 12.5, 14.5, or 17.5° 2θ±0.2° 2θ. In another embodiment, the tamibarotene:maleic acid crystalline form is characterized by a powder X-ray diffraction pattern comprising powder X-ray diffraction peaks at about 4.0, 6.0, 12.5, 14.5, and 17.5° 2θ±0.2° 2θ.

In another embodiment, the crystalline form is a tamibarotene:nicotinamide crystalline form. In one embodiment, the crystalline form of tamibarotene:nicotinamide is a 1:1 complex. In another embodiment, the tamibarotene:nicotinamide crystalline form is a co-crystal. In one embodiment, the tamibarotene:nicotinamide crystalline form is characterized by a powder X-ray diffraction pattern comprising a powder X-ray diffraction peak selected from about 4.0, 7.5, 14.5, 15.5, or 19.5° 2θ±0.2° 2θ. In another embodiment, the tamibarotene:nicotinamide crystalline form is characterized by a powder X-ray diffraction pattern comprising a powder X-ray diffraction peak at about 4.0° 2θ±0.2° 2θ. In another embodiment, the tamibarotene:nicotinamide crystalline form is characterized by a powder X-ray diffraction pattern comprising a powder X-ray diffraction peak at about 7.5° 2θ±0.2° 2θ. In another embodiment, the tamibarotene:nicotinamide crystalline form is characterized by a powder X-ray diffraction pattern comprising a powder X-ray diffraction peak at about 14.5° 2θ±0.2° 2θ. In another embodiment, the tamibarotene:nicotinamide crystalline form is characterized by a powder X-ray diffraction pattern comprising a powder X-ray diffraction peak at about 15.5° 2θ±0.2° 2θ. In another embodiment, the tamibarotene:nicotinamide crystalline form is characterized by a powder X-ray diffraction pattern comprising a powder X-ray diffraction peak at about 19.5° 2θ±0.2° 2θ. In another embodiment, the tamibarotene:nicotinamide crystalline form is characterized by a powder X-ray diffraction pattern comprising any four powder X-ray diffraction peaks selected from about 4.0, 7.5, 14.5, 15.5, or 19.5° 2θ±0.2° 2θ. In another embodiment, the tamibarotene:nicotinamide crystalline form is characterized by a powder X-ray diffraction pattern comprising any three powder X-ray diffraction peaks selected from about 4.0, 7.5, 14.5, 15.5, or 19.5° 2θ±0.2° 2θ. In another embodiment, the tamibarotene:nicotinamide crystalline form is characterized by a powder X-ray diffraction pattern comprising any two powder X-ray diffraction peaks selected from about 4.0, 7.5, 14.5, 15.5, or 19.5° 2θ±0.2° 2θ. In another embodiment, the tamibarotene:nicotinamide crystalline form is characterized by a powder X-ray diffraction pattern comprising powder X-ray diffraction peaks at about 4.0, 7.5, 14.5, 15.5, and 19.5° 2θ±0.2° 2θ.

In another embodiment, the crystalline form is a tamibarotene:isonicotinamide crystalline form. In one embodiment, the crystalline form of tamibarotene:isonicotinamide is a 1:1 complex. In another embodiment, the tamibarotene:isonicotinamide crystalline form is a co-crystal. In one embodiment, the tamibarotene:isonicotinamide crystalline form is characterized by a powder X-ray diffraction pattern comprising a powder X-ray diffraction peak selected from about 8.0, 9.0, 21.5, 22.0, or 24.0° 2θ±0.2° 2θ. In another embodiment, the tamibarotene:isonicotinamide crystalline form is characterized by a powder X-ray diffraction pattern comprising a powder X-ray diffraction peak at about 8.0° 2θ±0.2° 2θ. In another embodiment, the tamibarotene:isonicotinamide crystalline form is characterized by a powder X-ray diffraction pattern comprising a powder X-ray diffraction peak at about 9.0° 2θ±0.2° 2θ. In another embodiment, the tamibarotene:isonicotinamide crystalline form is characterized by a powder X-ray diffraction pattern comprising a powder X-ray diffraction peak at about 21.5° 2θ±0.2° 2θ. In another embodiment, the tamibarotene:isonicotinamide crystalline form is characterized by a powder X-ray diffraction pattern comprising a powder X-ray diffraction peak at about 22.0° 2θ±0.2° 2θ. In another embodiment, the tamibarotene:isonicotinamide crystalline form is characterized by a powder X-ray diffraction pattern comprising a powder X-ray diffraction peak at about 24.0° 2θ±0.2° 2θ. In another embodiment, the tamibarotene:isonicotinamide crystalline form is characterized by a powder X-ray diffraction pattern comprising any four powder X-ray diffraction peaks selected from about 8.0, 9.0, 21.5, 22.0, or 24.0° 2θ±0.2° 2θ. In another embodiment, the tamibarotene:isonicotinamide crystalline form is characterized by a powder X-ray diffraction pattern comprising any three powder X-ray diffraction peaks selected from about 8.0, 9.0, 21.5, 22.0, or 24.0° 2θ±0.2° 2θ. In another embodiment, the tamibarotene:isonicotinamide crystalline form is characterized by a powder X-ray diffraction pattern comprising any two powder X-ray diffraction peaks selected from about 8.0, 9.0, 21.5, 22.0, or 24.0° 2θ±0.2° 2θ. In another embodiment, the tamibarotene:isonicotinamide crystalline form is characterized by a powder X-ray diffraction pattern comprising powder X-ray diffraction peaks at about 8.0, 9.0, 21.5, 22.0, and 24.0° 2θ±0.2° 2θ.

In another embodiment, the crystalline form is a tamibarotene:citric acid crystalline form. In one embodiment, the crystalline form of tamibarotene:citric acid is a 1:1 complex. In another embodiment, the tamibarotene; citric acid crystalline form is a co-crystal. In one embodiment, the tamibarotene:citric acid crystalline form is characterized by a powder X-ray diffraction pattern comprising a powder X-ray diffraction peak selected from about 6.5, 8.5, 12.5, 16.5, or 19.5° 2θ±0.2° 2θ. In another embodiment, the tamibarotene:citric acid crystalline form is characterized by a powder X-ray diffraction pattern comprising a powder X-ray diffraction peak at about 6.5° 2θ±0.2° 2θ. In another embodiment, the tamibarotene:citric acid crystalline form is characterized by a powder X-ray diffraction pattern comprising a powder X-ray diffraction peak at about 8.5° 2θ±0.2° 2θ. In another embodiment, the tamibarotene:citric acid crystalline form is characterized by a powder X-ray diffraction pattern comprising a powder X-ray diffraction peak at about 12.5° 2θ±0.2° 2θ. In another embodiment, the tamibarotene:citric acid crystalline form is characterized by a powder X-ray diffraction pattern comprising a powder X-ray diffraction peak at about 16.5° 2θ±0.2° 2θ. In another embodiment, the tamibarotene:citric acid crystalline form is characterized by a powder X-ray diffraction pattern comprising a powder X-ray diffraction peak at about 19.5° 2θ±0.2° 2θ. In another embodiment, the tamibarotene:citric acid crystalline form is characterized by a powder X-ray diffraction pattern comprising any four powder X-ray diffraction peaks selected from about 6.5, 8.5, 12.5, 16.5, or 19.5° 2θ±0.2° 2θ. In another embodiment, the tamibarotene:citric acid crystalline form is characterized by a powder X-ray diffraction pattern comprising any three powder X-ray diffraction peaks selected from about 6.5, 8.5, 12.5, 16.5, or 19.5° 2θ±0.2° 2θ. In another embodiment, the tamibarotene:citric acid crystalline form is characterized by a powder X-ray diffraction pattern comprising any two powder X-ray diffraction peaks selected from about 6.5, 8.5, 12.5, 16.5, or 19.5° 2θ±0.2° 2θ. In another embodiment, the tamibarotene:citric acid crystalline form is characterized by a powder X-ray diffraction pattern comprising powder X-ray diffraction peaks at about 6.5, 8.5, 12.5, 16.5, and 19.5° 2θ±0.2° 2θ.

In another one embodiment, the crystalline form is a tamibarotene:nicotinic acid crystalline form. In one embodiment, the crystalline form of tamibarotene:nicotinic acid is a 1:1 complex. In another embodiment, the tamibarotene:nicotinic acid crystalline form is a co-crystal. In one embodiment, the tamibarotene:nicotinic acid crystalline form is characterized by a powder X-ray diffraction pattern comprising a powder X-ray diffraction peak selected from about 6.5, 8.5, 12.5, 16.5, 19.0, or 19.5° 2θ±0.2° 2θ. In another embodiment, the tamibarotene:nicotinic acid crystalline form is characterized by a powder X-ray diffraction pattern comprising a powder X-ray diffraction peak at about 6.5° 2θ±0.2° 2θ. In another embodiment, the tamibarotene:nicotinic acid crystalline form is characterized by a powder X-ray diffraction pattern comprising a powder X-ray diffraction peak at about 8.5° 2θ±0.2° 2θ. In another embodiment, the tamibarotene:nicotinic acid crystalline form is characterized by a powder X-ray diffraction pattern comprising a powder X-ray diffraction peak at about 12.5° 2θ±0.2° 2θ. In another embodiment, the tamibarotene:nicotinic acid crystalline form is characterized by a powder X-ray diffraction pattern comprising a powder X-ray diffraction peak at about 16.5° 2θ±0.2° 2θ. In another embodiment, the tamibarotene:nicotinic acid crystalline form is characterized by a powder X-ray diffraction pattern comprising a powder X-ray diffraction peak at about 19.0° 2θ±0.2° 2θ. In another embodiment, the tamibarotene:nicotinic acid crystalline form is characterized by a powder X-ray diffraction pattern comprising a powder X-ray diffraction peak at about 19.5° 2θ±0.2° 2θ. In another embodiment, the tamibarotene:nicotinic acid crystalline form is characterized by a powder X-ray diffraction pattern comprising any five powder X-ray diffraction peaks selected from about 6.5, 8.5, 12.5, 16.5, 19.0, or 19.5° 2θ±0.2° 2θ. In another embodiment, the tamibarotene:nicotinic acid crystalline form is characterized by a powder X-ray diffraction pattern comprising any four powder X-ray diffraction peaks selected from about 6.5, 8.5, 12.5, 16.5, 19.0, or 19.5° 2θ±0.2° 2θ. In another embodiment, the tamibarotene:nicotinic acid crystalline form is characterized by a powder X-ray diffraction pattern comprising any three powder X-ray diffraction peaks selected from about 6.5, 8.5, 12.5, 16.5, 19.0, or 19.5° 2θ±0.2° 2θ. In another embodiment, the tamibarotene:nicotinic acid crystalline form is characterized by a powder X-ray diffraction pattern comprising any two powder X-ray diffraction peaks selected from about 6.5, 8.5, 12.5, 16.5, 19.0, or 19.5° 2θ±0.2° 2θ. In another embodiment, the tamibarotene:nicotinic acid crystalline form is characterized by a powder X-ray diffraction pattern comprising powder X-ray diffraction peaks at about 6.5, 8.5, 12.5, 16.5, 19.0, and 19.5° 2θ±0.2° 2θ.

In another embodiment, the crystalline form is a tamibarotene:3,4-dihydroxybenzoic acid crystalline form. In one embodiment, the crystalline form of tamibarotene:3,4-dihydroxybenzoic acid is a 1:1 complex. In another embodiment, the tamibarotene:3,4-dihydroxybenzoic acid crystalline form is a co-crystal. In one embodiment, the tamibarotene:3,4-dihydroxybenzoic acid crystalline form is characterized by a powder X-ray diffraction pattern comprising a powder X-ray diffraction peak selected from about 4.5, 9.5, 18.5, 23.5, or 24.5° 2θ±0.2° 2θ. In another embodiment, the tamibarotene:3,4-dihydroxybenzoic acid crystalline form is characterized by a powder X-ray diffraction pattern comprising a powder X-ray diffraction peak at about 4.5° 2θ±0.2° 2θ. In another embodiment, the tamibarotene:3,4-dihydroxybenzoic acid crystalline form is characterized by a powder X-ray diffraction pattern comprising a powder X-ray diffraction peak at about 9.5° 2θ±0.2° 2θ. In another embodiment, the tamibarotene:3,4-dihydroxybenzoic acid crystalline form is characterized by a powder X-ray diffraction pattern comprising a powder X-ray diffraction peak at about 18.5° 2θ±0.2° 2θ. In another embodiment, the tamibarotene:3,4-dihydroxybenzoic acid crystalline form is characterized by a powder X-ray diffraction pattern comprising a powder X-ray diffraction peak at about 23.5° 2θ±0.2° 2θ. In another embodiment, the tamibarotene:3,4-dihydroxybenzoic acid crystalline form is characterized by a powder X-ray diffraction pattern comprising a powder X-ray diffraction peak at about 24.5° 2θ±0.2° 2θ. In another embodiment, the tamibarotene:3,4-dihydroxybenzoic acid crystalline form is characterized by a powder X-ray diffraction pattern comprising any four powder X-ray diffraction peaks selected from about 4.5, 9.5, 18.5, 23.5, or 24.5° 2θ±0.2° 2θ. In another embodiment, the tamibarotene:3,4-dihydroxybenzoic acid crystalline form is characterized by a powder X-ray diffraction pattern comprising any three powder X-ray diffraction peaks selected from about 4.5, 9.5, 18.5, 23.5, or 24.5° 2θ±0.2° 2θ. In another embodiment, the tamibarotene:3,4-dihydroxybenzoic acid crystalline form is characterized by a powder X-ray diffraction pattern comprising any two powder X-ray diffraction peaks selected from about 4.5, 9.5, 18.5, 23.5, or 24.5° 2θ±0.2° 2θ. In another embodiment, the tamibarotene:3,4-dihydroxybenzoic acid crystalline form is characterized by a powder X-ray diffraction pattern comprising powder X-ray diffraction peaks at about 4.5, 9.5, 18.5, 23.5, and 24.5° 2θ±0.2° 2θ.

In another embodiment, the crystalline form is a tamibarotene:glutaric acid crystalline form. In one embodiment, the crystalline form of tamibarotene:glutaric acid is a 1:1 complex. In another embodiment, the tamibarotene:glutaric acid crystalline form is a co-crystal. In one embodiment, the tamibarotene:glutaric acid crystalline form is characterized by a powder X-ray diffraction pattern comprising a powder X-ray diffraction peak selected from about 3.5, 7.0, 8.5, 14.5, or 21.5° 2θ±0.2° 2θ. In another embodiment, the tamibarotene:glutaric acid crystalline form is characterized by a powder X-ray diffraction pattern comprising a powder X-ray diffraction peak at about 3.5° 2θ±0.2° 2θ. In another embodiment, the tamibarotene:glutaric acid crystalline form is characterized by a powder X-ray diffraction pattern comprising a powder X-ray diffraction peak at about 7.0° 2θ±0.2° 2θ. In another embodiment, the tamibarotene:glutaric acid crystalline form is characterized by a powder X-ray diffraction pattern comprising a powder X-ray diffraction peak at about 8.5° 2θ±0.2° 2θ. In another embodiment, the tamibarotene:glutaric acid crystalline form is characterized by a powder X-ray diffraction pattern comprising a powder X-ray diffraction peak at about 14.5° 2θ±0.2° 2θ. In another embodiment, the tamibarotene:glutaric acid crystalline form is characterized by a powder X-ray diffraction pattern comprising a powder X-ray diffraction peak at about 21.5° 2θ±0.2° 2θ. In another embodiment, the tamibarotene:glutaric acid crystalline form is characterized by a powder X-ray diffraction pattern comprising any four powder X-ray diffraction peaks selected from about 3.5, 7.0, 8.5, 14.5, or 21.5° 2θ±0.2° 2θ. In another embodiment, the tamibarotene:glutaric acid crystalline form is characterized by a powder X-ray diffraction pattern comprising any three powder X-ray diffraction peaks selected from about 3.5, 7.0, 8.5, 14.5, or 21.5° 2θ±0.2° 2θ. In another embodiment, the tamibarotene:glutaric acid crystalline form is characterized by a powder X-ray diffraction pattern comprising any two powder X-ray diffraction peaks selected from about 3.5, 7.0, 8.5, 14.5, or 21.5° 2θ±0.2° 2θ. In another embodiment, the tamibarotene:glutaric acid crystalline form is characterized by a powder X-ray diffraction pattern comprising powder X-ray diffraction peaks at about 3.5, 7.0, 8.5, 14.5, and 21.5° 2θ±0.2° 2θ.

In another embodiment, the crystalline form is a tamibarotene:L-malic acid crystalline form. In one embodiment, the crystalline form of tamibarotene:L-malic acid is a 1:1 complex. In another embodiment, the tamibarotene:L-malic acid crystalline form is a co-crystal. In one embodiment, the tamibarotene:L-malic acid crystalline form is characterized by a powder X-ray diffraction pattern comprising a powder X-ray diffraction peak selected from about 10.5, 12.0, 14.0, 19.5, or 24.5° 2θ±0.2° 2θ. In another embodiment, the tamibarotene:L-malic acid crystalline form is characterized by a powder X-ray diffraction pattern comprising a powder X-ray diffraction peak at about 10.5° 2θ±0.2° 2θ. In another embodiment, the tamibarotene:L-malic acid crystalline form is characterized by a powder X-ray diffraction pattern comprising a powder X-ray diffraction peak at about 12.0° 2θ±0.2° 2θ. In another embodiment, the tamibarotene:L-malic acid crystalline form is characterized by a powder X-ray diffraction pattern comprising a powder X-ray diffraction peak at about 14.0° 2θ±0.2° 2θ. In another embodiment, the tamibarotene:L-malic acid crystalline form is characterized by a powder X-ray diffraction pattern comprising a powder X-ray diffraction peak at about 19.5° 2θ±0.2° 2θ. In another embodiment, the tamibarotene:L-malic acid crystalline form is characterized by a powder X-ray diffraction pattern comprising a powder X-ray diffraction peak at about 24.5° 2θ±0.2° 2θ. In another embodiment, the tamibarotene:L-malic acid crystalline form is characterized by a powder X-ray diffraction pattern comprising any four powder X-ray diffraction peaks selected from about 10.5, 12.0, 14.0, 19.5, or 24.5° 2θ±0.2° 2θ. In another embodiment, the tamibarotene:L-malic acid crystalline form is characterized by a powder X-ray diffraction pattern comprising any three powder X-ray diffraction peaks selected from about 10.5, 12.0, 14.0, 19.5, or 24.5° 2θ±0.2° 2θ. In another embodiment, the tamibarotene:L-malic acid crystalline form is characterized by a powder X-ray diffraction pattern comprising any two powder X-ray diffraction peaks selected from about 10.5, 12.0, 14.0, 19.5, or 24.5° 2θ±0.2° 2θ. In another embodiment, the tamibarotene:L-malic acid crystalline form is characterized by a powder X-ray diffraction pattern comprising powder X-ray diffraction peaks selected at 10.5, 12.0, 14.0, 19.5, and 24.5° 2θ±0.2° 2θ.

The present invention includes complexes of tamibarotene with adipic acid, or DL-aspartic acid, or acetylsalicylic acid, or biphenyl-4-carboxylic acid, or caffeic acid, or decanoic acid, or diphenic acid, or gallic acid, or fumaric acid, or ibuprofen, or maleic acid, or nicotinamide, or isonicotinamide, or citric acid, or nicotinic acid, or 3,4-dihydroxybenzoic acid, or glutaric acid, or L-malic acid, which are capable of complexing in the solid-state for example, through dry or solvent-drop grinding, heating or solvent evaporation of their solution in single or mixed solvent systems, slurry suspension, antisolvent, supercritical fluids, or other techniques known to a person skilled in the art. Solvents and antisolvents used to make the crystalline forms include acetone, ethanol, methanol, ethylacetate (EtOAc), isopropanol (IP A), or isopropylacetate (IP Ac), diethoxymethane (DEM), toluene, BuOAc, N-methylpyrrolidone (NMP), and a heptane.

In one embodiment, the invention includes crystalline forms of tamibarotene and adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, or L-malic acid, which are capable of complexing through solvent evaporation of their solution in single or mixed solvent systems, and slurry suspension.

In another embodiment, the invention includes crystalline forms of tamibarotene with adipic acid, or DL-aspartic acid, or acetylsalicylic acid, or biphenyl-4-carboxylic acid, or caffeic acid, or decanoic acid, or diphenic acid, or gallic acid, or fumaric acid, or ibuprofen, or maleic acid, or nicotinamide, or isonicotinamide, or citric acid, or nicotinic acid, or 3,4-dihydroxybenzoic acid, or glutaric acid, or L-malic acid, which have shown physical stability during storage under accelerated conditions of temperature of 40° C. and 75% relative humidity for at least one year.

In another embodiment of the invention includes crystalline forms of tamibarotene with or biphenyl-4-carboxylic acid, or diphenic acid, or gallic acid, or ibuprofen, or nicotinamide, or isonicotinamide or glutaric acid, which have shown physical stability during storage under accelerated conditions of temperature of 40° C. and 75% relative humidity for at least one year.

In another embodiment, the molecular complex of tamibarotene and adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, or L-malic acid can be scaled at least 10×.

In another embodiment, the molecular complex of tamibarotene and biphenyl-4-carboxylic acid, decanoic acid, diphenic acid, gallic acid, ibuprofen, nicotinamide, or glutaric acid can be scaled up at least 10×.

In another embodiment, the complex of tamibarotene and biphenyl-4-carboxylic acid, decanoic acid, diphenic acid, gallic acid, or nicotinamide have shown anti-cancer activity measured by the half-maximal inhibitory concentration (IC₅₀).

In another embodiment, the complex of tamibarotene and biphenyl-4-carboxylic acid, decanoic acid, diphenic acid, gallic acid, or nicotinamide have shown improved (IC₅₀) compared with the parent molecule tamibarotene in treating sarcoma, skin, prostate, and pancreatic cancer.

In another embodiment, the complex of tamibarotene with gallic acid has improved (IC₅₀) by two orders of magnitude compared with the parent molecule.

In another embodiment, the novel molecular complex tamibarotene with gallic acid has reduced tumor sphere formation efficiency compared with the parent molecule.

In another embodiment, the novel molecular complex tamibarotene with gallic acid has reduced tumor spheres size compared with that of the parent molecule.

In another aspect, the invention provides for a pharmaceutical composition comprising a molecular complex of the present invention. In one embodiment, the molecular complex is a crystalline form. In a further embodiment, the crystalline form is a crystalline form of tamibarotene and adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, or L-malic acid. In another embodiment, the crystalline form is a cocrystal of tamibarotene and adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, or L-malic acid.

The pharmaceutical composition comprises a therapeutically effective amount of at least one of the novel molecular complexes of tamibarotene according to the invention and at least one pharmaceutically acceptable excipient. The term “therapeutically effective amount” means an amount of active ingredients (e.g., tamibarotene: coformer) that will elicit a desired biological or pharmacological response, e.g., effective to prevent, alleviate, or ameliorate symptoms of a disorder or prolong the survival of the subject being treated. The term “excipient” refers to a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for intravenous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A pharmaceutical formulation of the present invention may be in any pharmaceutical dosage form. The pharmaceutical formulation may be, for example, a tablet, capsule, nanoparticulate material, e.g., granulated particulate material or a powder, a lyophilized material for reconstitution, liquid suspension, injectable suspension or solution, suppository, or topical or transdermal preparation or patch. The pharmaceutical formulations generally contain about 1% to about 99% by weight of at least one novel molecular complex of tamibarotene of the invention and 99% to 1% by weight of a suitable pharmaceutical excipient. In one embodiment, the dosage form is an oral dosage form. In another embodiment, the dosage form is a parenteral dosage form. In one embodiment, the pharmaceutical dosage form is a unit dose. The term “unit dose” refers to the amount of API, e.g., tamibarotene:former, administered to a patient in a single dose.

The novel molecular complexes of tamibarotene are therapeutically useful for the treatment, prevention, and/or cure of a disease for which it is indicated, e.g., cancer. Accordingly, in another aspect, the invention also relates a method of treating or preventing a disease for which tamibarotene is indicated, the method comprising the step of administering to a patient in need thereof, a therapeutically effective amount of a pharmaceutical composition of the present invention.

In some embodiments, a pharmaceutical composition of the present invention is delivered to a subject via intratumoral injection. “Intratumoral injection” is a route of administration by which a pharmaceutical composition is delivered directly to the tumor via an injection device (e.g., needle and syringe). In other embodiments, a pharmaceutical composition of the present invention is delivered to a subject via a parenteral route, an enteral route, or a topical route.

Examples of parental routes the present invention include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous.

Enteral routes of administration of the present invention include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories.

Topical, including transdermal, routes of administration of the present invention include administration to a body surface, such as skin or mucous membranes. Delivery vehicles of the present disclosure may be administered topically (or transdermally) via a cream, foam, gel, lotion or ointment, for example.

As used herein, the terms “treat,” “treating,” or “treatment” means to alleviate, reduce or abrogate one or more symptoms or characteristics of a disease and may be curative, palliative, prophylactic or slow the progression of the disease. The term “subject” or “patient” includes mammals, especially humans. In one embodiment, the patient is a human. In another embodiment, the patient is a human male. In another embodiment, the patient is a human female. In another embodiment, the patient is a warm-blooded mammal.

In one embodiment, the invention provides for a method of treating a patient suffering from a disease or condition for which tamibarotene is indicated, the method comprising the step of administering to the patient a therapeutically effective amount of a pharmaceutical composition of the present invention.

In another embodiment, the disease or condition is selected from acute promyelocytic leukaemia (APL), Alzheimer's disease, multiple myeloma and Crohn's disease, systemic lupus erythematosus (SLE), cutaneous lupus erythematosus (CLE), drug-induced lupus, neutropenia, neonatal lupus, and rheumatoid arthritis.

In another embodiment, the invention provides for a method of treating pre-cancer or cancer comprising the step of administering to a pre-cancer or cancer patient a therapeutically effective amount of a pharmaceutical composition of the present invention. The present invention further provides for a medicament comprising a pharmaceutical composition of the present invention for use in treating pre-cancer or cancer. In one embodiment, the cancer is acute promyelocytic leukaemia (APL).

In another embodiment, the invention provides for a method of inhibiting the growth of cancer stem cells in a cancer patient, the method comprising the step of administering to the cancer patient a therapeutically effective amount of a pharmaceutical composition of the present invention.

In another embodiment, the invention provides for a method of treating neutropenia, inhibiting neutropenia, reducing the severity of neutropenia, or promoting neutropenia prophylaxis, the method comprising the step of administering a therapeutically effective amount of a pharmaceutical composition of the present invention to a patient suffering from neutropenia or at risk of developing neutropenia.

In another embodiment, the invention provides for a composition for reducing an apparent effect of skin aging, the composition comprising a tamibarotene molecular complex of the present invention in a cosmetically acceptable carrier.

In another embodiment, the invention provides for a method of reducing an apparent effect of skin aging in a patient in need thereof, comprising the step of applying a topical formulation comprising a tamibarotene molecular complex of the present invention in a cosmetically acceptable carrier. In one embodiment, the apparent effect of aging is selected from wrinkles, folds, pigmented spots, or dry skin.

The dosage may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In some embodiments, the cancer is selected from: acute promyelocytic leukaemia (APL), Wilms' tumor, rhabdomyosarcoma, ovarian cancer (e.g., germ cell), gestational trophoblastic neoplasm, Ewing's sarcoma, metastatic testicular tumors (e.g., nonseminoatous), gestational trophoblastic neoplasm, locally recurrent or locoregional solid tumors (sarcomas, carcinomas and adenocarcinomas), acute myeloid leukemia (AML), prostate cancer, skin cancer, actinic keratosis, Bowen's disease, adjuvant cancer therapy, or neoadjuvant cancer therapy. In a preferred embodiment, the cancer is skin cancer, actinic keratosis, or Bowen's disease. In a further embodiment, the skin cancer is selected from the group consisting of: basal cell carcinoma (BCC), squamous cell carcinoma (SCC), and melanoma. In another embodiment, the cancer is prostate cancer. In a further embodiment, the prostate cancer is selected from the group consisting of: acinar adenocarcinoma, ductal adenocarcinoma, transitional cell (or urothelial) cancer, squamous cell cancer, small cell prostate cancer, carcinoid, and sarcoma.

### EXAMPLES

The techniques and approaches set forth in the present disclosure can further be used by the person of ordinary skill in the art to prepare variants thereof, said variants are considered to be part of the present invention.

Materials used to create the novel forms of the present inventions are commercially available and means to synthesize them as well known. Tamibarotene as a starting material used in all experiments in this disclosure was supplied by Selleck Chemicals Inc., Houston, Tex., USA with >99% purity. All other pure chemicals (Analytical Grade) were purchased from available commercial sources and used as purchased.

Solid Phase Characterization

Analytical techniques used to observe the crystalline forms include powder X-ray diffraction (PXRD) and Fourier transform infrared spectroscopy (FTIR). The particular methodology used in such analytical techniques should be viewed as illustrative, and not limiting in the context of data collection. For example, the particular instrumentation used to collect data may vary; routine operator error or calibration standards may vary; sample preparation method may vary (for example, the use of the KBr disk or Nujol mull technique for FTIR analysis).

Fourier Transform FTIR Spectroscopy (FTIR): FTIR analysis was performed on a Perkin Elmer Spectrum 100 FTIR spectrometer equipped with a solid-state ATR accessory.

Powder X-Ray Diffraction (PXRD): All tamibarotene novel molecular complex products were observed by a D-8 Bruker X-ray Powder Diffractometer using Cu Kα (λ=1.540562 Λ), 40 kV, 40 mA. The data were collected over an angular range of 3° to 40° 2θ in continuous scan mode at room temperature using a step size of 0.05° 2θ and a scan speed of 6.17°/min.

The following examples illustrate the invention without intending to limit the scope of the invention.

### Example 1: Preparation of Tamibarotene:Adipic Acid Complex

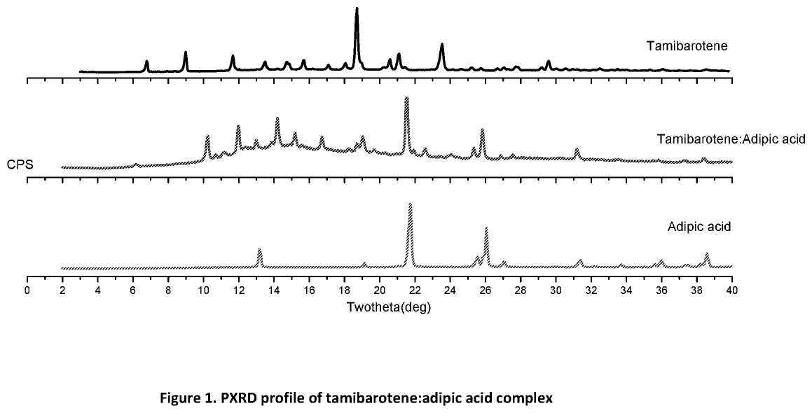

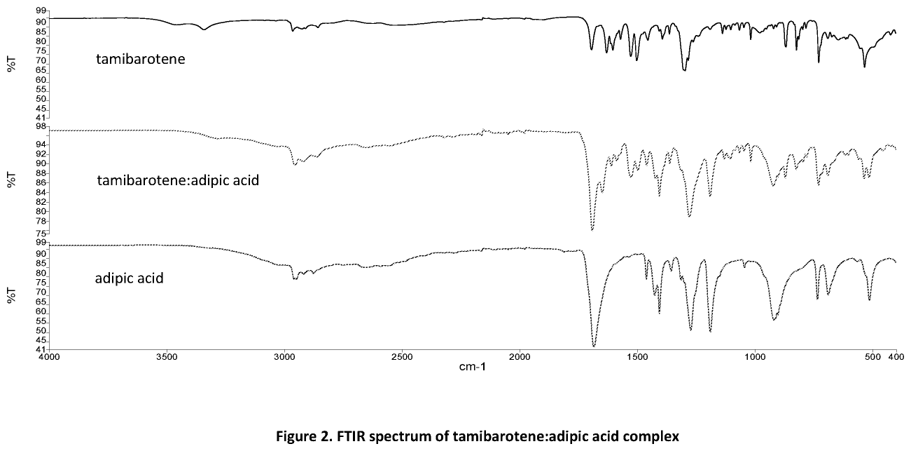

50 mg of recrystallized tamibarotene in acetonitrile and 20 mg of adipic acid (1:1 molar ratio) was stirred as a slurry in an open 20 mL glass vial with 1 mL of acetone. After 12-16 hours the stirring was stopped, and the mixture was dried at room temperature for another 12-16 hours. The solids gathered were dried and stored in a screw cap vials for subsequent analysis. The material was characterized by PXRD and FTIR corresponding to FIGS. 1 and 2, respectively.

### Example 2: Preparation of Tamibarotene:DL-Aspartic Acid Complex

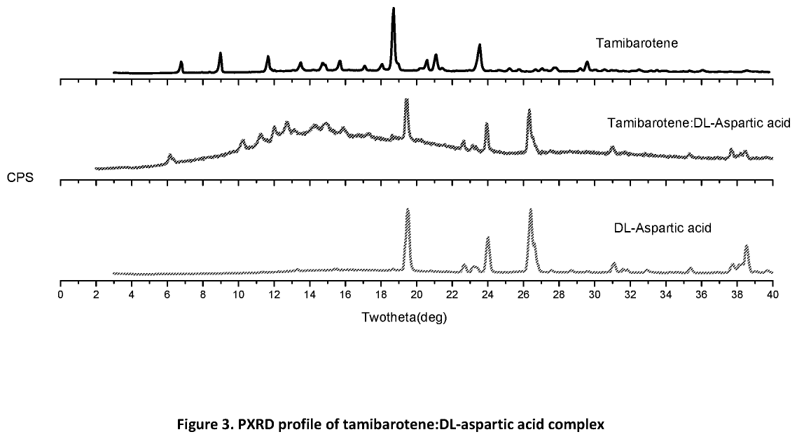

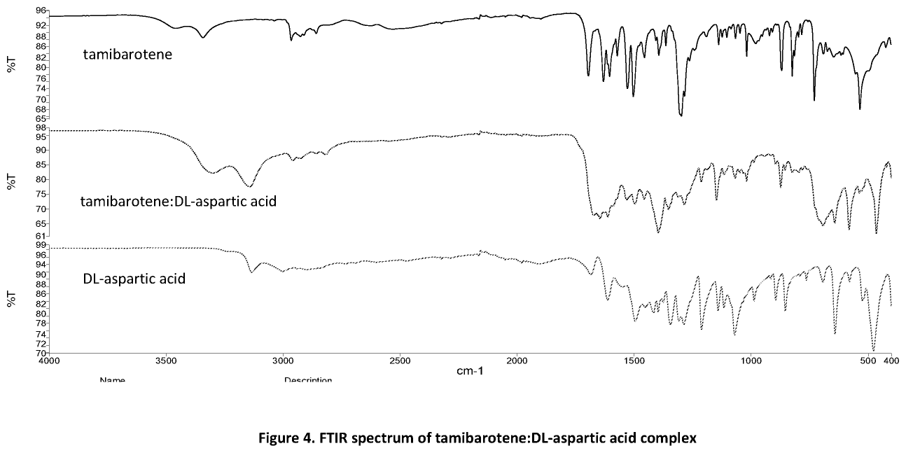

50 mg of recrystallized tamibarotene in acetonitrile and 16.5 mg of DL-aspartic acid (1:1 molar ratio) was stirred as a slurry in an open 20 mL glass vial with 1 mL of acetone. After 12-16 hours the stirring was stopped, and mixture was dried at room temperature for another 12-16 hours. Solids were dried and stored in a screw cap vials for subsequent analysis. All materials were characterized by PXRD and FTIR corresponding to FIGS. 3 and 4, respectively.

### Example 3: Preparation of Tamibarotene:Acetylsalicylic Acid Complex

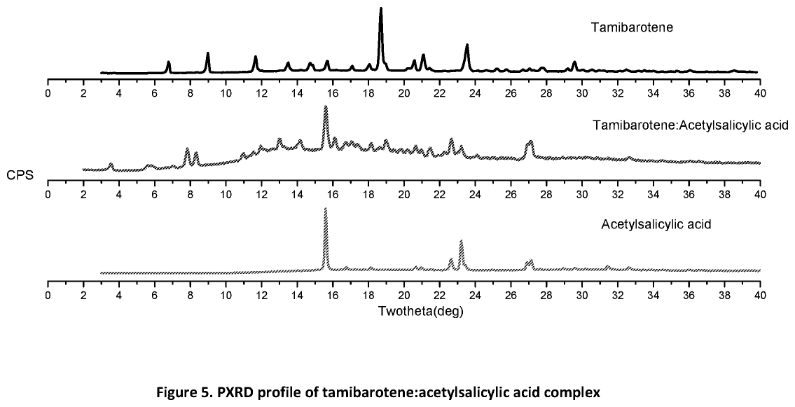

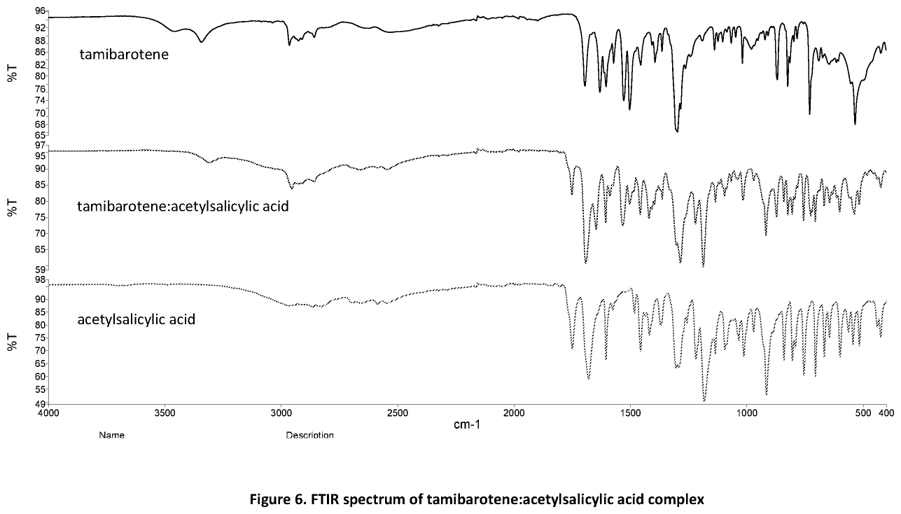

50 mg of recrystallized tamibarotene in acetonitrile and 26 mg of acetylsalicylic acid (1:1 molar ratio) was stirred as a slurry in an open 20 mL borosilicate glass scintillation vial with 1 mL of acetone. After 12-16 hours the stirring was stopped, and mixture was dried at room temperature for another 12-16 hours. The material was stored for subsequent analysis and characterized by PXRD and FTIR corresponding to FIGS. 5 and 6, respectively.

### Example 4: Preparation of Tamibarotene:Biphenyl-4-Carboxylic Acid Complex

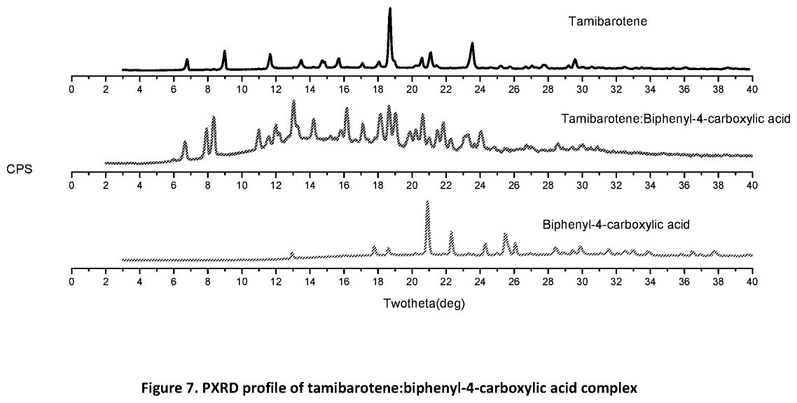

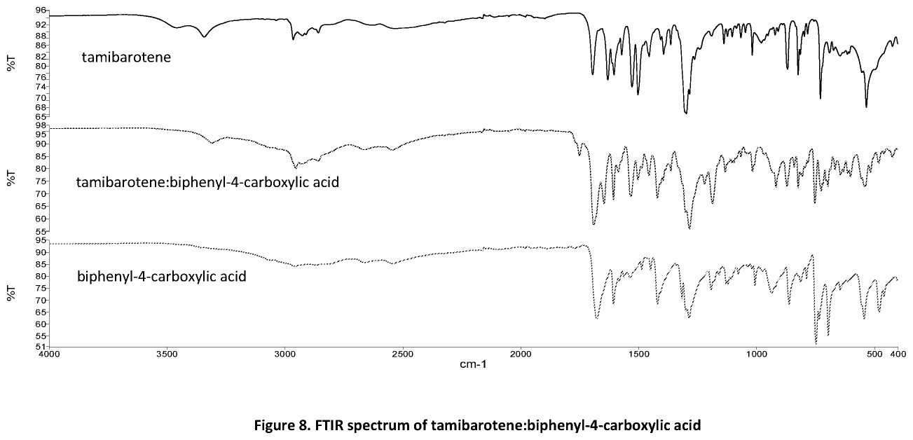

50 mg of recrystallized tamibarotene in acetonitrile and 28 mg of biphenyl-4-carboxylic acid (1:1 molar ratio) was stirred as a slurry in an open 20 mL glass scintillation vial with 1 mL of acetone. After 12-16 hours the stirring was stopped, and mixture was dried at room temperature for another 12-16 hours. The material was stored for subsequent analysis and characterized by PXRD and FTIR corresponding to FIGS. 7 and 8, respectively.

### Example 5: Preparation of Tamibarotene:Caffeic Acid Complex

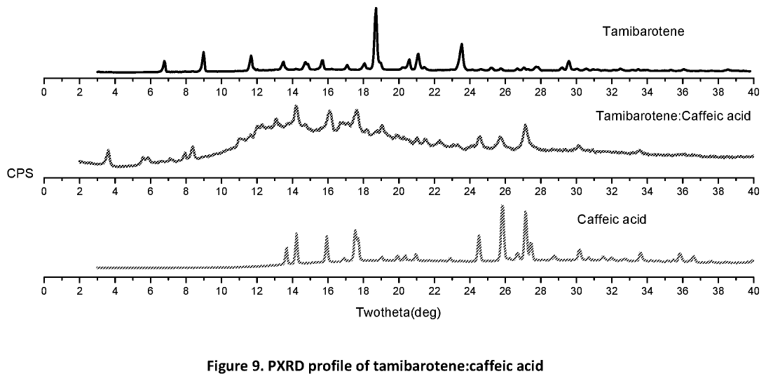

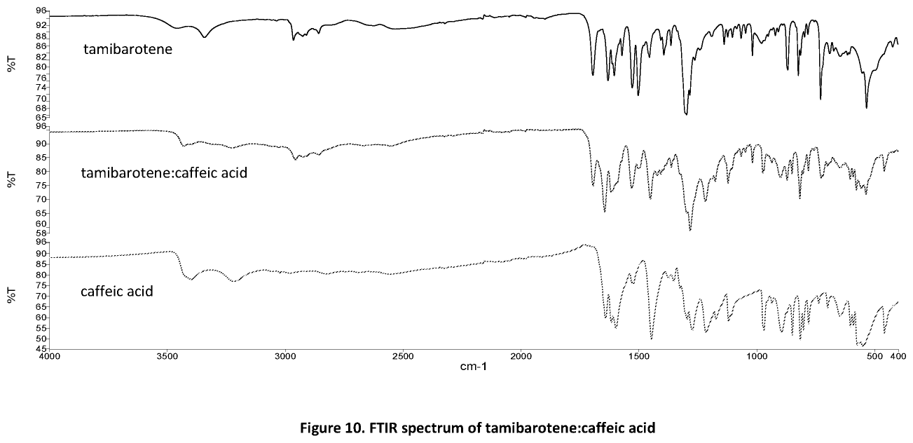

50 mg of recrystallized tamibarotene in acetonitrile and 26 mg of caffeic acid (1:1 molar ratio) was stirred as a slurry in an open 20 mL glass scintillation vial with 1 mL of acetone. After 12-16 hours the stirring was stopped, and mixture was dried at room temperature for another 12-16 hours. The solids were dried and stored in a screw cap vials for subsequent analysis. The material was characterized by PXRD and FTIR corresponding to FIGS. 9 and 10, respectively.

### Example 6: Preparation of Tamibarotene:Decanoic Acid Complex

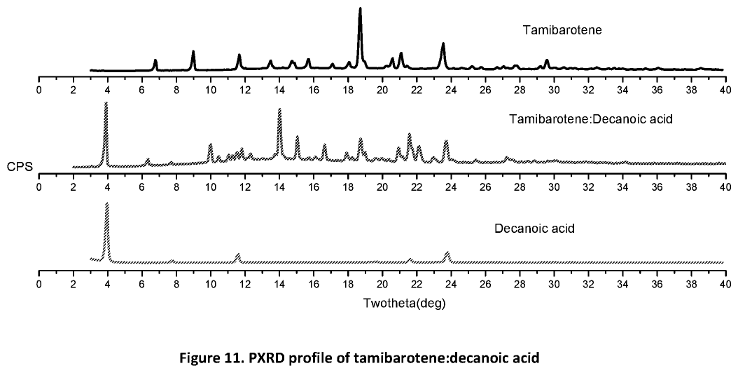

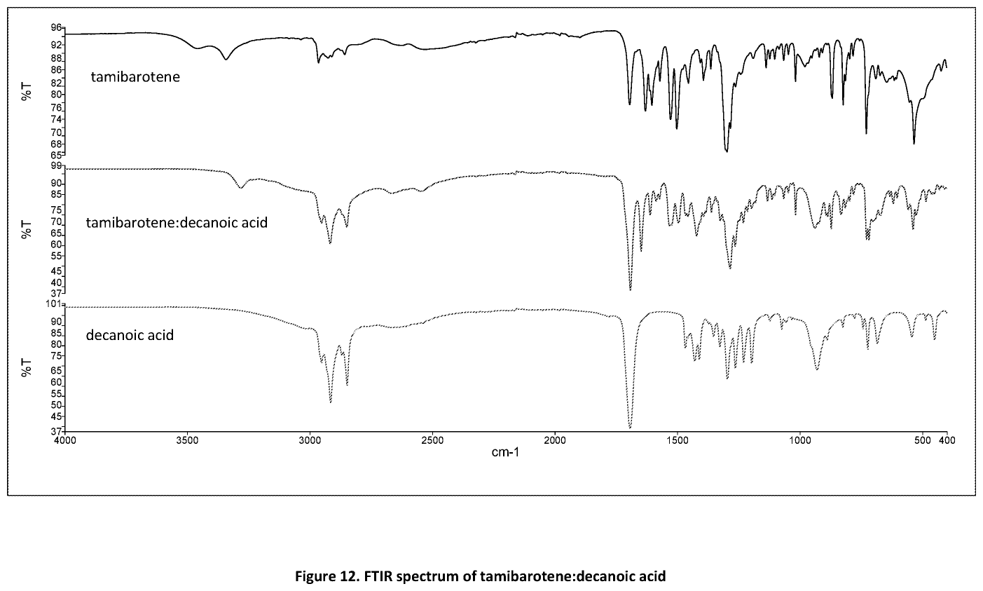

50 mg of recrystallized tamibarotene in acetonitrile and 24.5 mg of decanoic acid (1:1 molar ratio) was stirred as a slurry in an open 20 mL glass scintillation vial with 1 mL of acetone. After 12-16 hours the stirring was stopped, and mixture was dried at room temperature for another 12-16 hours. The solids were dried and stored in a screw cap vials for subsequent analysis. The material was characterized by PXRD and FTIR corresponding to FIGS. 11 and 12, respectively.

### Example 7: Preparation of Tamibarotene:Diphenic Acid Complex

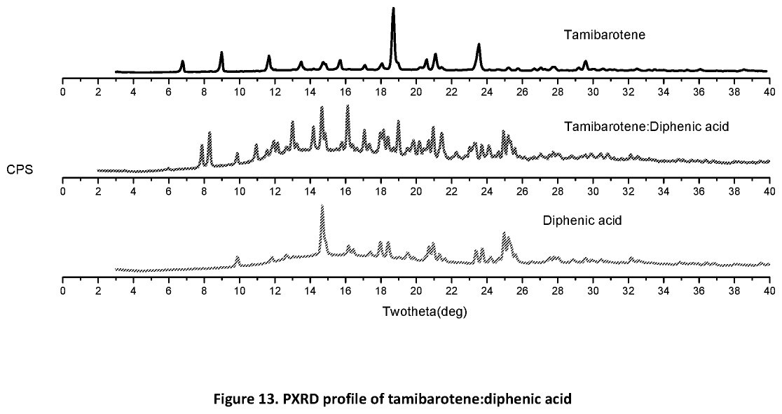

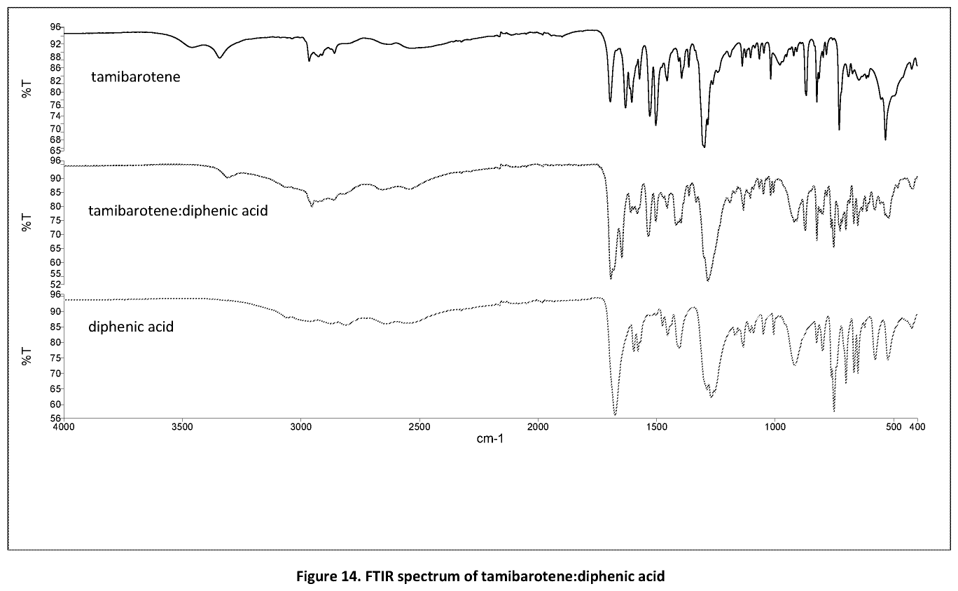

50 mg of recrystallized tamibarotene in acetonitrile and 34.4 mg of diphenic acid (1:1 molar ratio) was stirred as a slurry in an open 20 mL borosilicate glass scintillation vial with 1 mL of acetone. After 12-16 hours the stirring was stopped, and mixture was dried at room temperature for another 12-16 hours. Resulted solids were dried and stored in screw cap vials and characterized by PXRD and FTIR corresponding to FIGS. 13 and 14, respectively.

### Example 8: Preparation of Tamibarotene:Gallic Acid Complex

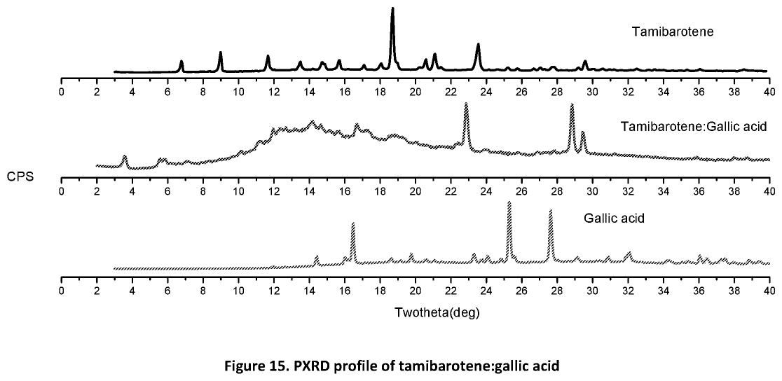

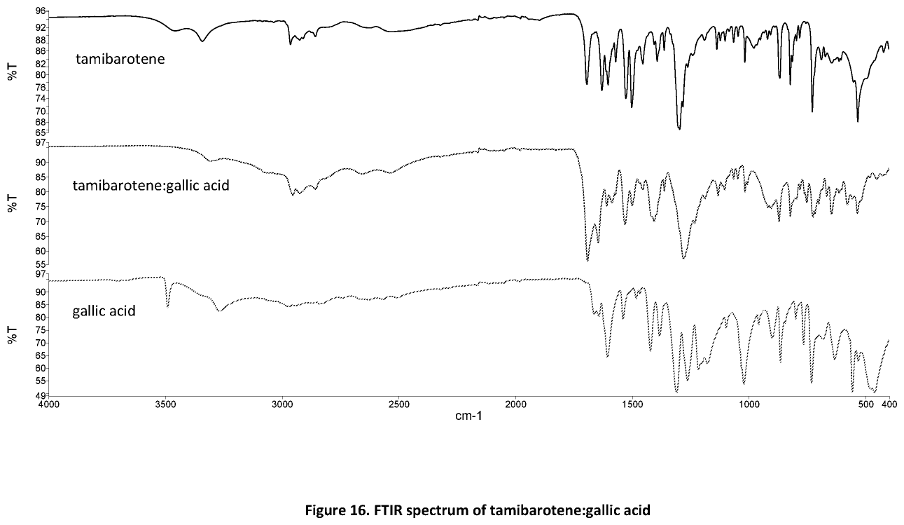

50 mg of tamibarotene and 24 mg of gallic acid (1:1 molar ratio) was stirred as a slurry in an open 20 mL glass scintillation vial with 1 mL of acetone. After 12-16 hours the stirring was stopped, and mixture was dried at room temperature for another 12-16 hours. Resulted solids were dried and stored in screw cap vials and characterized by PXRD and FTIR corresponding to FIGS. 15 and 16, respectively.

### Example 9: Preparation of Tamibarotene:Fumaric Acid Complex

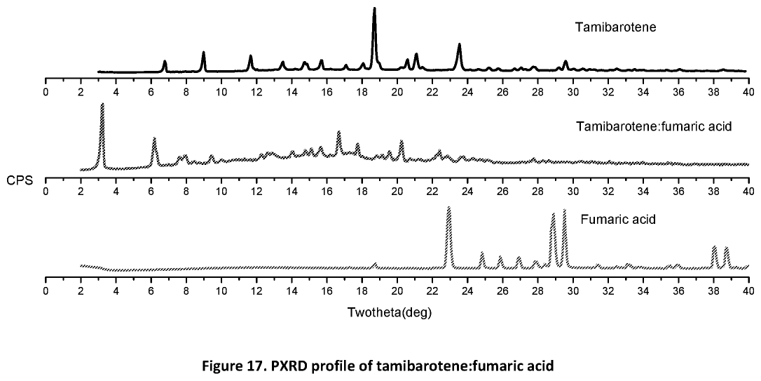

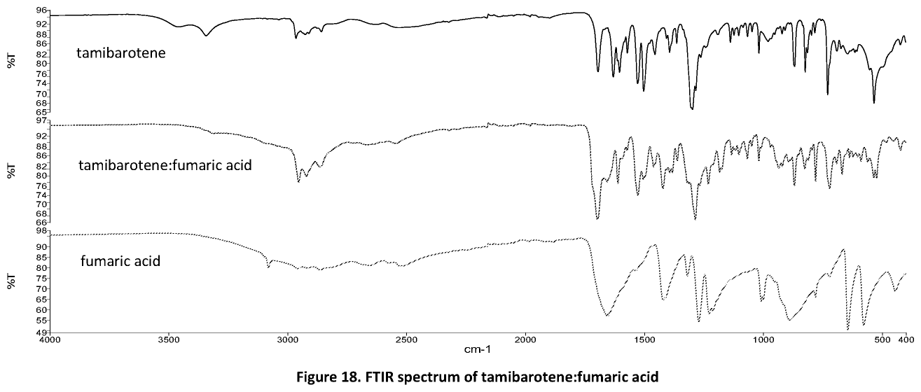

50 mg of tamibarotene and 16.5 mg of fumaric acid (1:1 molar ratio) was stirred as a slurry in an open 20 mL glass scintillation vial with 1 mL of acetone. After 12-16 hours the stirring was stopped, and mixture was dried at room temperature for another 12-16 hours. Resulted solids were dried and stored in screw cap vials and characterized by PXRD and FTIR corresponding to FIGS. 17 and 18, respectively.

### Example 10: Preparation of Tamibarotene:Ibuprofen Complex

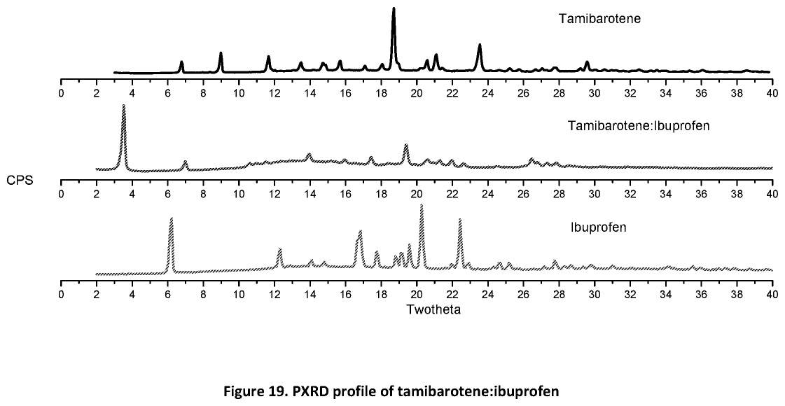

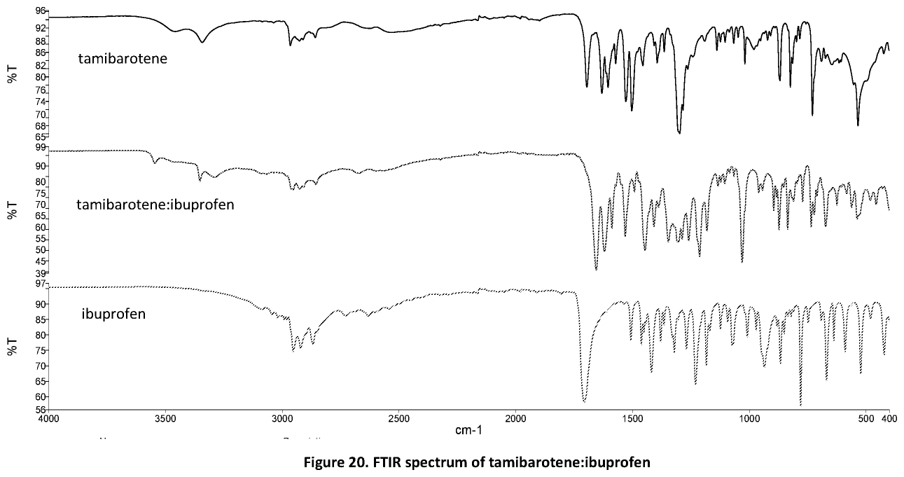

50 mg of recrystallized tamibarotene in acetonitrile and 29 mg of ibuprofen (1:1 molar ratio) was stirred as a slurry in an open 20 mL glass scintillation vial with 1 mL of acetone. After 12-16 hours the stirring was stopped, and mixture was dried at room temperature for another 12-16 hours. Resulted solids were dried and stored in screw cap vials and characterized by PXRD and FTIR corresponding to FIGS. 19 and 20, respectively.

### Example 11: Preparation of Tamibarotene:Maleic Acid Complex

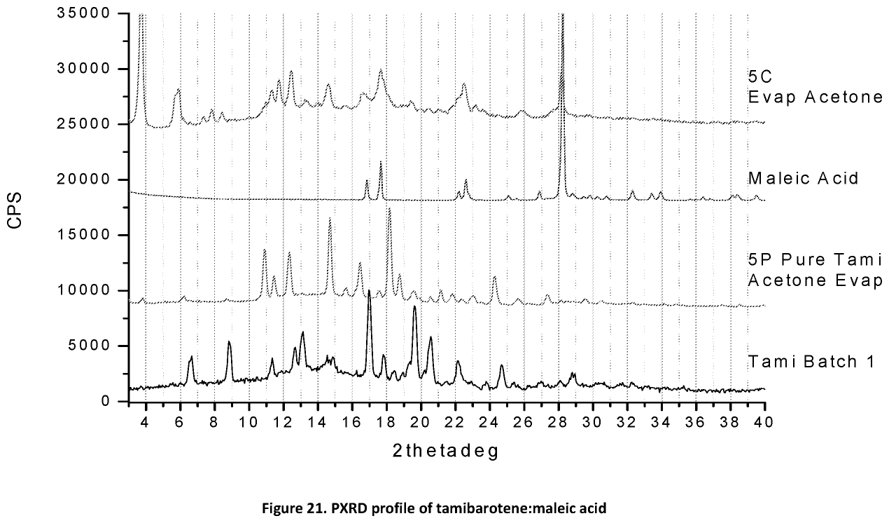

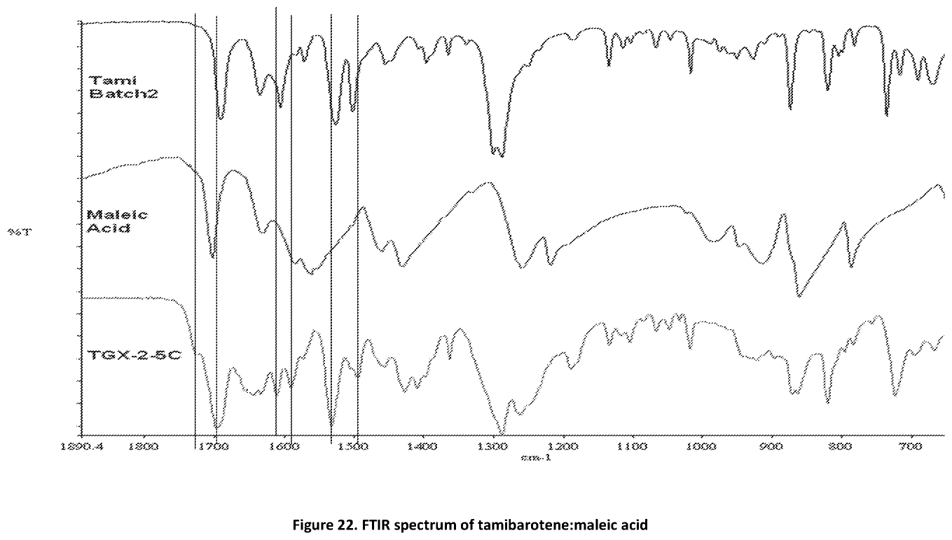

70 mg of tamibarotene and 22 mg of maleic acid was dissolved in 1 mL of acetone in a 20 mL glass vial and let evaporate at 25° C. until dry. Resulted solids were stored in screw cap vials and characterized by PXRD and FTIR corresponding to FIGS. 21 and 22, respectively.

### Example 12: Preparation of Tamibarotene:Nicotinamide Complex

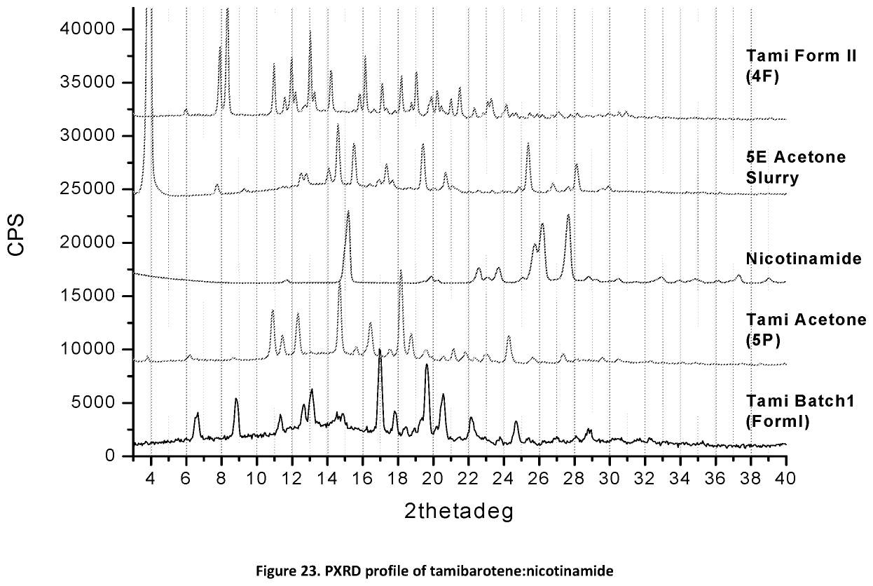

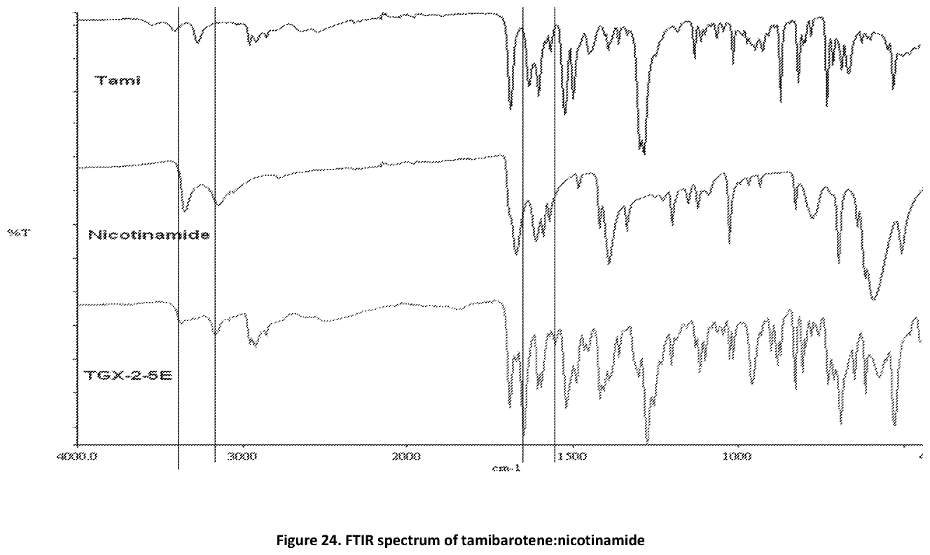

70 mg of tamibarotene and 23 mg of nicotinamide were stirred as a slurry in a small glass vial with 1 mL of acetone for 12-16 hours and the solids filtered off. The material was air dried for 16-24 hours. The solids were collected and stored in screw cap vials and characterized by PXRD and FTIR corresponding to FIGS. 23 and 24, respectively.

### Example 13: Preparation of Tamibarotene:Isonicotinamide Complex

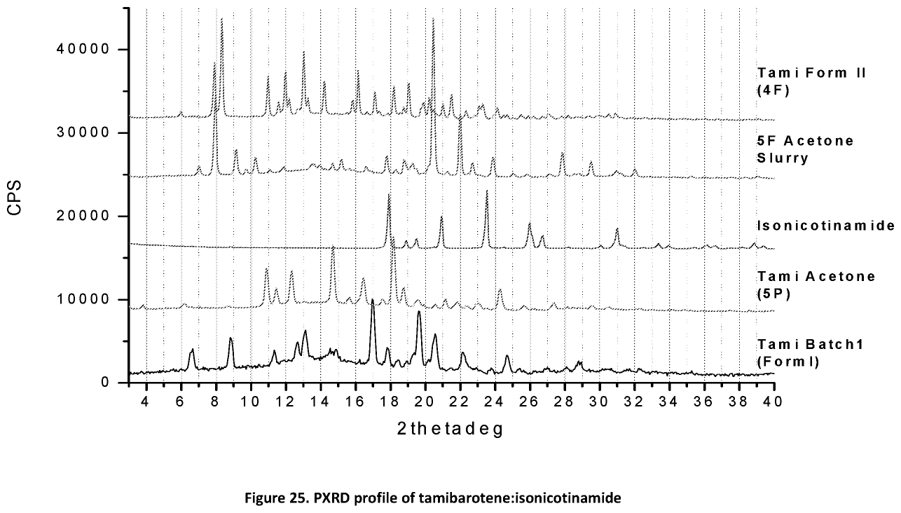

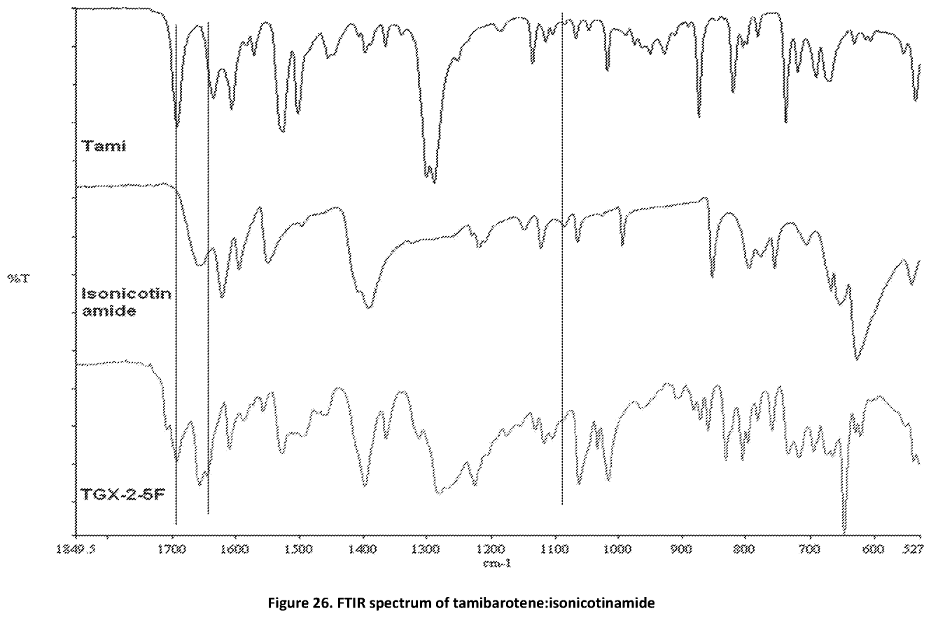

70 mg of tamibarotene and 23 mg of isonicotinamide were stirred as a slurry in a small glass vial with 1 mL of acetone for 12-16 hours and the solids filtered off. The material was air dried for 16-24 hours. The solids were collected and stored in screw cap vials and characterized by PXRD and FTIR corresponding to FIGS. 25 and 26, respectively.

### Example 14: Preparation of Tamibarotene:Citric Acid Complex

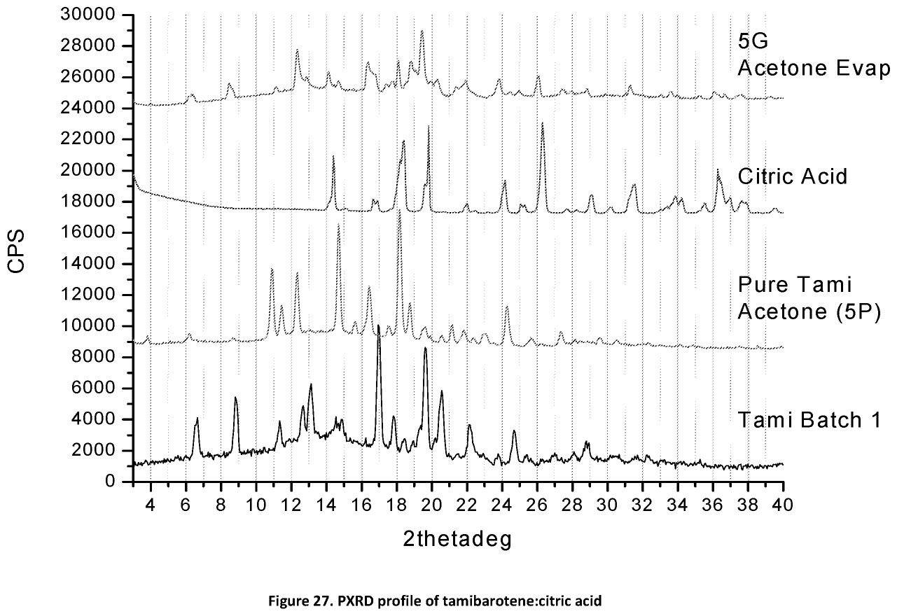

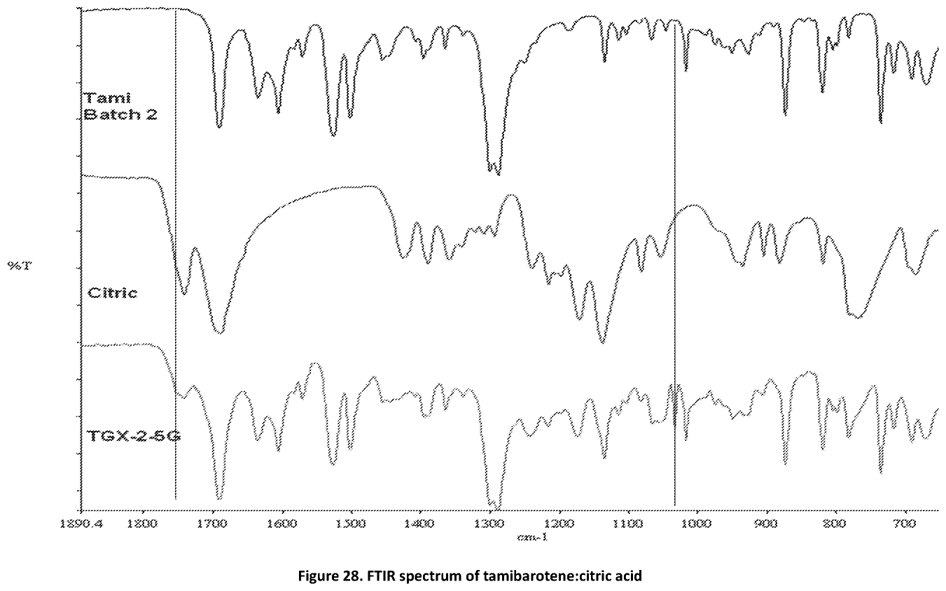

50 mg of tamibarotene and 27.3 mg of citric acid were dissolved in 1 mL of acetone and let evaporate at 25° C. until dry. The solids were collected and stored in screw cap vials and characterized by PXRD and FTIR corresponding to FIGS. 27 and 28, respectively.

### Example 15: Preparation of Tamibarotene:Nicotinic Acid Complex

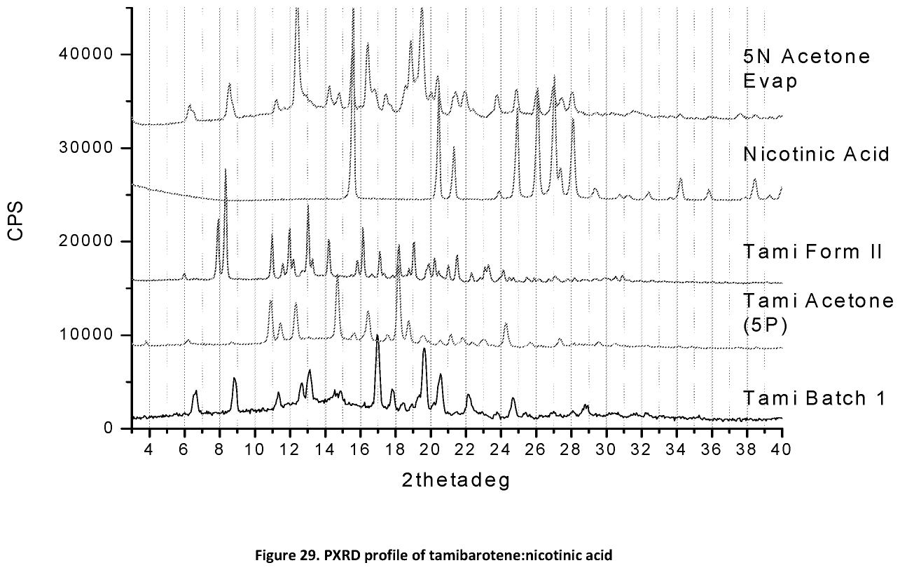

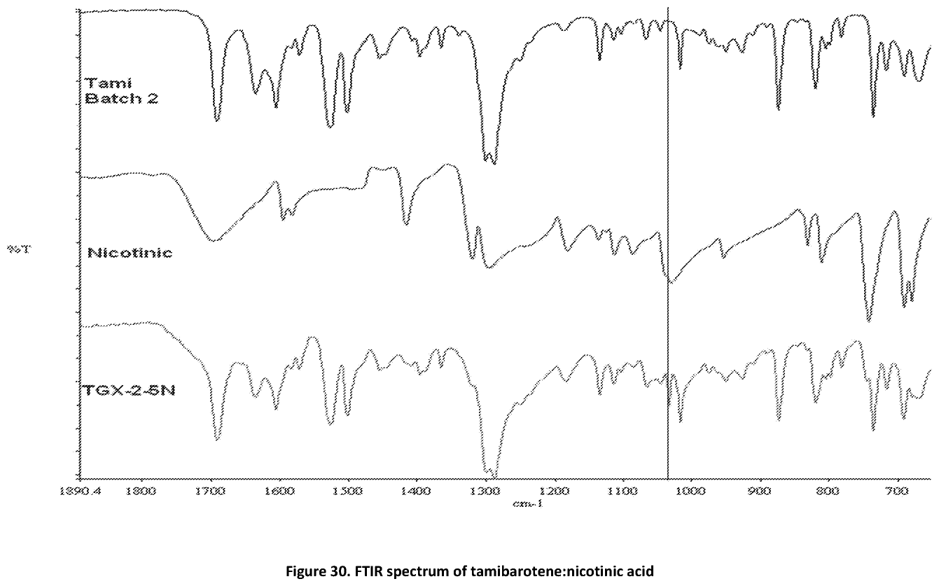

50 mg of tamibarotene and 16.7 mg of nicotinic acid were dissolved in 3.5 mL of 2:5 acetone:methanol and let evaporate at 25° C. until dry. The solids were collected and stored in screw cap vials and characterized by PXRD and FTIR corresponding to FIGS. 29 and 30, respectively.

### Example 16: Preparation of Tamibarotene:3,4-Dihydroxybenzoic Acid Complex

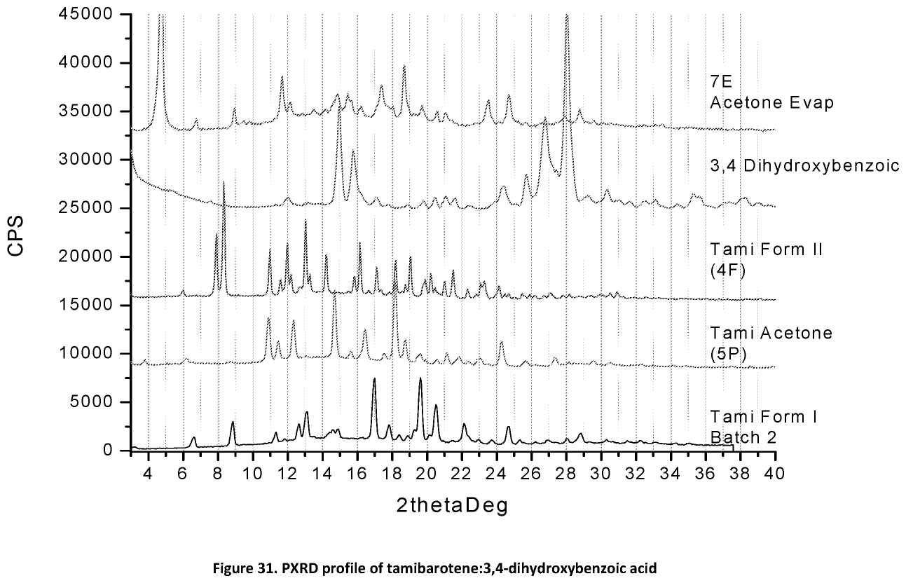

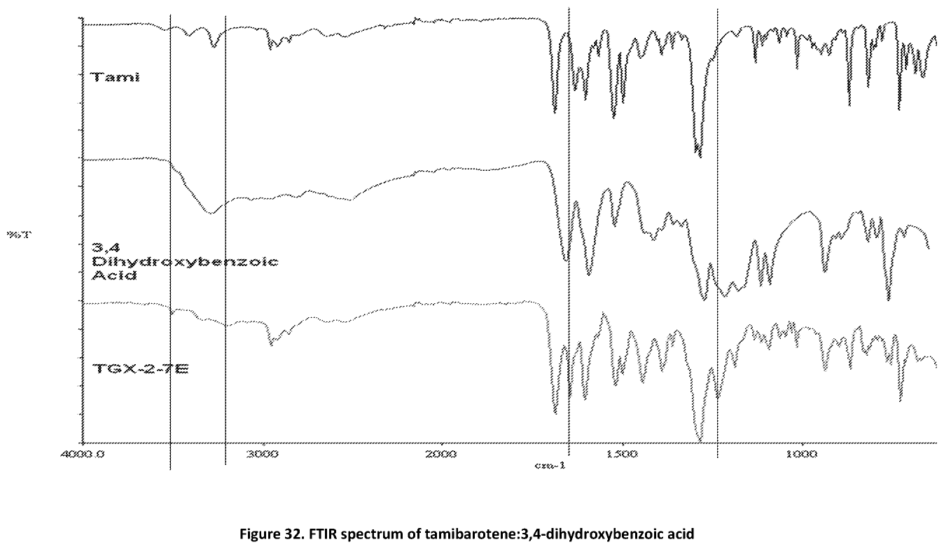

A thin cloudy suspension was created by adding 1 mL of acetone to 50 mg of tamibarotene and 9.4 mg of 3,4-dihydroxybenzoic acid and let evaporate at 25° C. until dry. The resulting solids were stored in screw cap vials and characterized by PXRD and FTIR corresponding to FIGS. 31 and 32, respectively.

### Example 17: Preparation of Tamibarotene:Glutaric Acid Complex

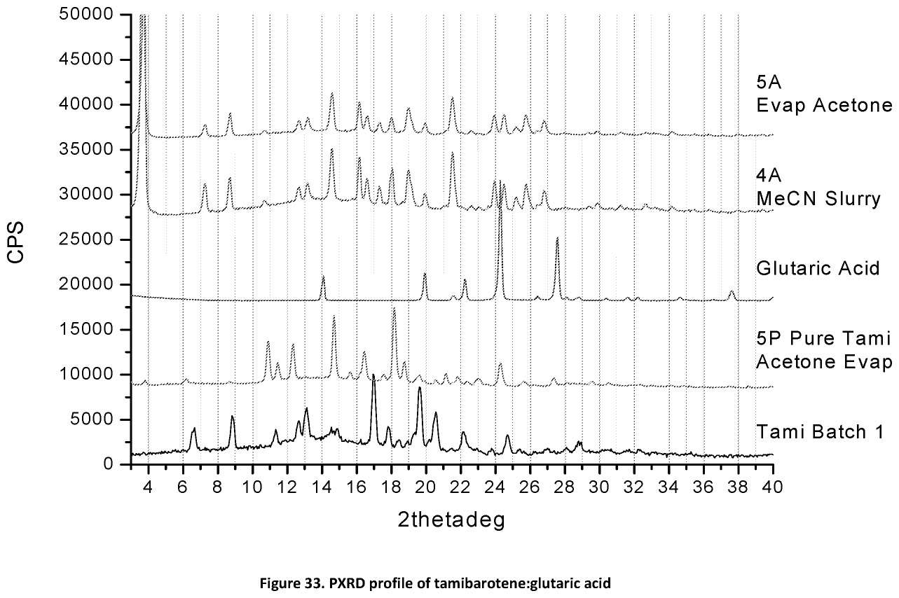

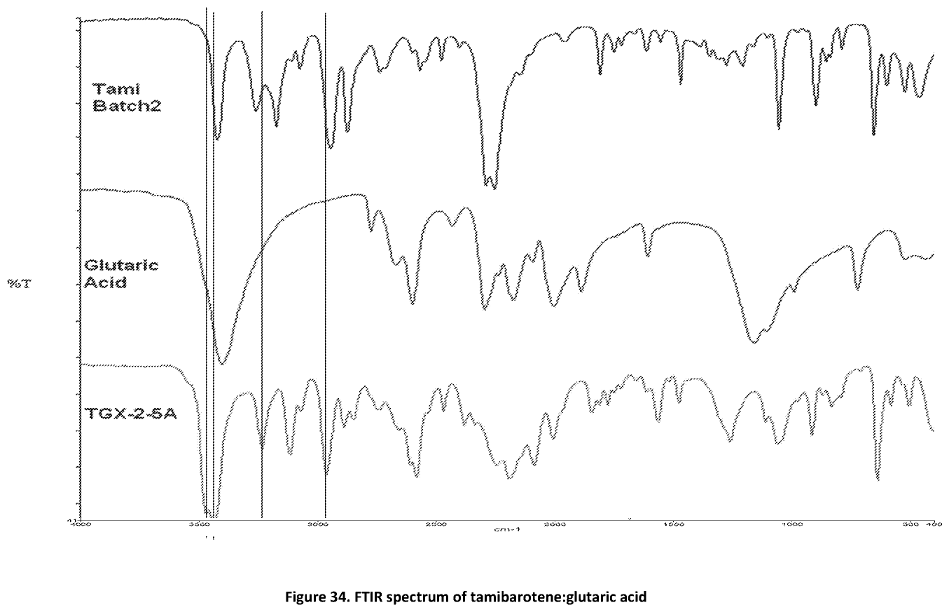

50 mg of tamibarotene and 25 mg of glutaric acid were stirred as a slurry in a small glass vial with 5.2 mL of acetonitrile for 12-16 hours and filter off the solid. The material was air dried for 16-24 hours. In an alternative method, the same amount of both molecules was dissolved in 1 mL of acetone and let evaporate at 25° C. until dry. The resulting solids were stored in screw cap vials and characterized by PXRD and FTIR corresponding to FIGS. 33 and 34, respectively.

### Example 18: Preparation of Tamibarotene:L-Malic Acid Complex

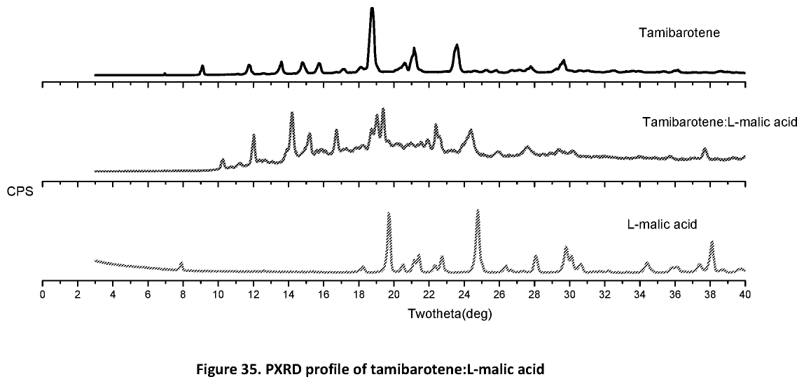

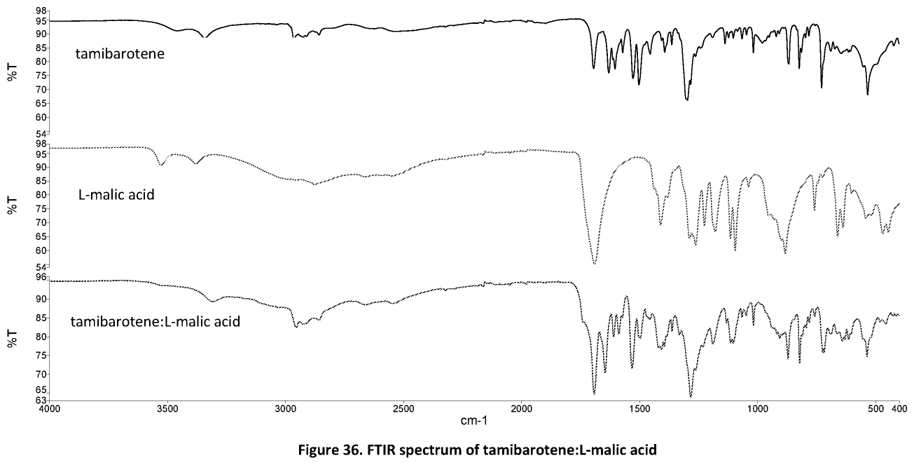

70 mg of tamibarotene and 25.4 mg of L-malic acid were dissolved in 1 mL of acetone and let evaporate at 25° C. until dry. The resulting solids were stored in screw cap vials and characterized by PXRD and FTIR corresponding to FIGS. 35 and 36, respectively.

### Example 19: Scale Up Experiments

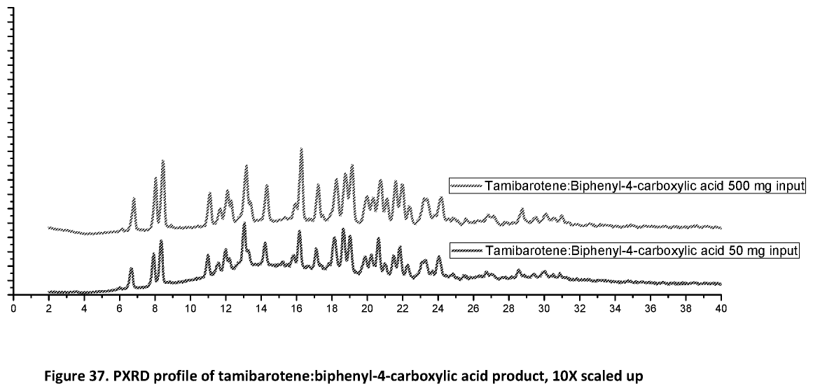

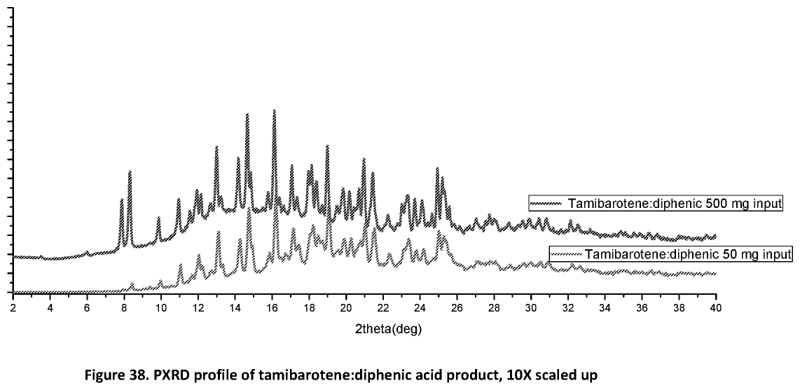

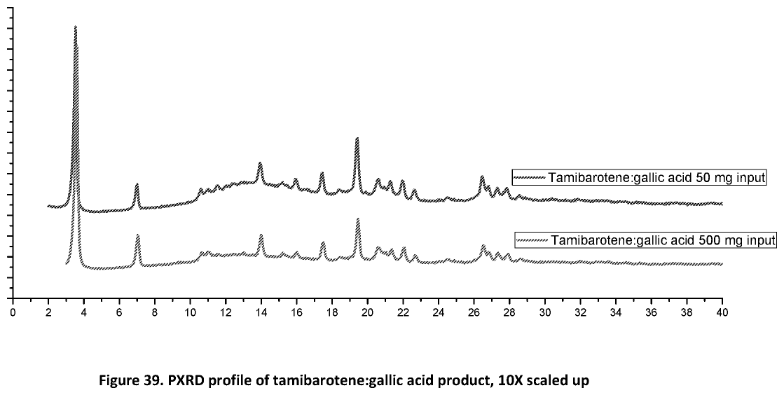

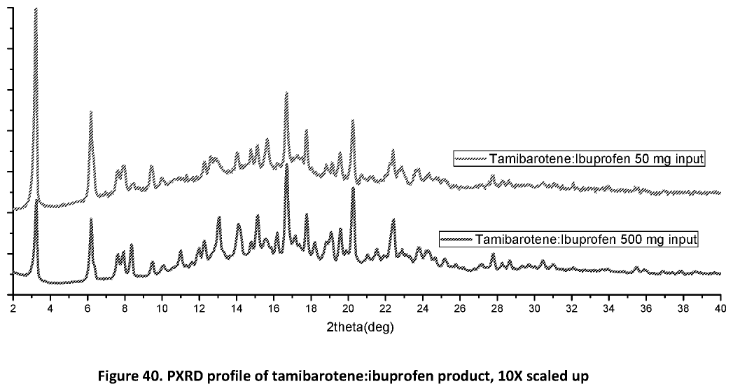

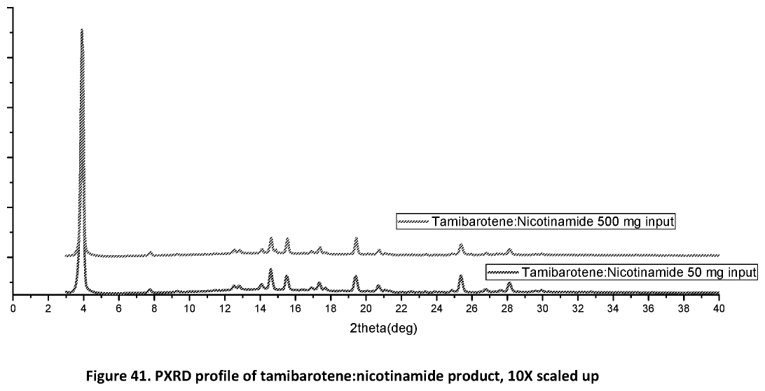

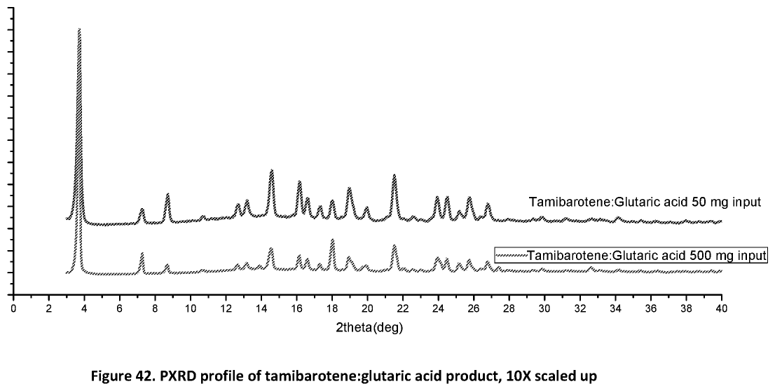

This mg level synthesis was successfully scaled up to 10× level product to demonstrate scalability of this process. Scale up experiments were successfully carried out for a gram level for tamibarotene molecular complex products; from 50 mg tamibarotene input to 500 mg input tamibarotene. Examples of the molecular complexes efficaciously generated include but not limited to tamibarotene:biphenyl-4-carboxilic acid, tamibarotene:diphenic acid, tamibarotene:gallic acid, tamibarotene:ibuprofen, tamibarotene:nicotinamide, tamibarotene:glutaric acid as verified by PXRD profiles in FIG. 37-42 of this disclosure that shows that the 10× scaled up product has the same diffractogram as that of the small scale experiment.

### Example 20: Accelerated Stability Studies

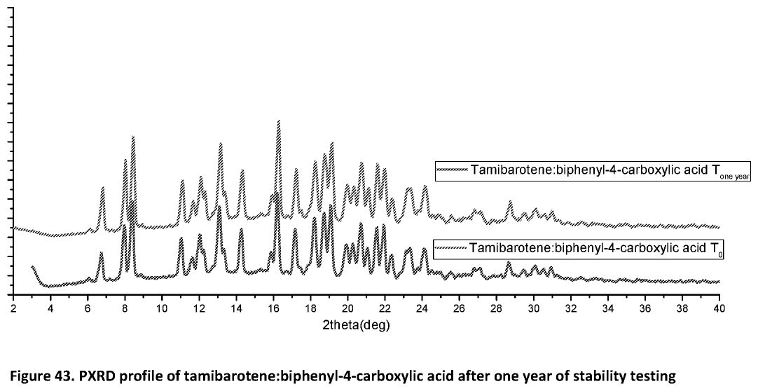

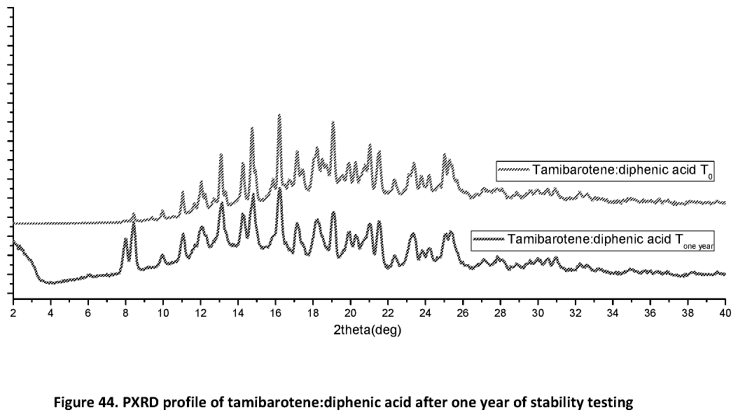

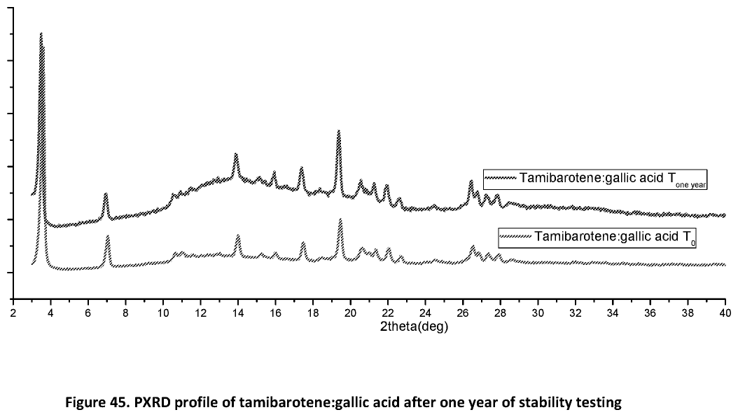

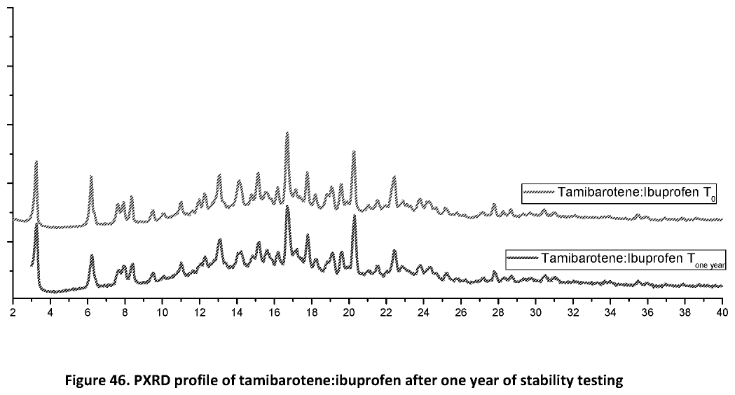

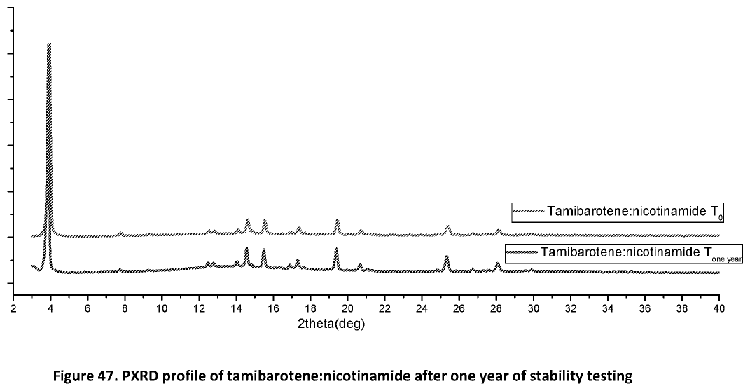

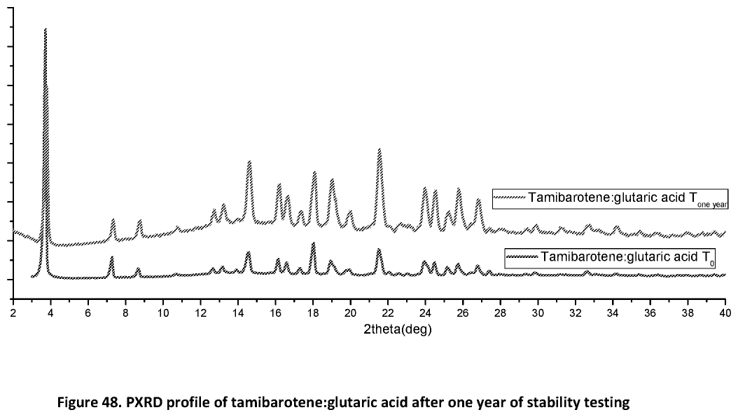

Stability studies of the novel tamibarotene generated molecular complexes conducted under accelerated conditions (75% humidity and 40° C.), which are obvious to the person skilled in the art of physical pharmaceutics. The novel complexes were stressed under heat and humidity for over a year and proved to be physically stable. Samples of selected forms were pulled for physical testing and characterization at intervals of 1, 3, 6, and 12 months. PXRD data of the novel forms including tamibarotene:biphenyl-4-carboxilic acid, tamibarotene:diphenic acid, tamibarotene:gallic acid, tamibarotene:ibuprofen, tamibarotene:nicotinamide, tamibarotene:glutaric acid has shown that the those novel molecular complexes were physically stable even after 12 months of storage under accelerated conditions. See FIG. 43-48.

### Example 21: IC₅₀ Studies

Cancer cell lines listed in Table 1, were subjected for testing anti-cancer potency or the half maximal inhibitory concentration (IC₅₀) of tamibarotene molecular complexes.

TABLE 1
Cell lines used for test on anti-cancer function of the molecular complexes

| Cell Name | Catalog Name | Disease Type | Original Tissue |
|---|---|---|---|
| SK-ES-1 | ATCC ® HTB-86 | anaplastic osteosarcoma or | bone |
| | | Ewing's sarcoma | |
| U-2 OS | ATCC ® HTB-96 | osteosarcoma | bone |
| SK-MEL-5 | ATCC ® HTB-70 | malignant melanoma | skin: derived from metastatic |
| | | | axillary node |
| PC3 | ATCC ® CRL-1435 | adenocarcinoma | prostate; derived from |
| | | | metastatic site: bone |
| PANC-1 | ATCC ® CRL-1435 | epithelioid carcinoma | pancreas/duct |
| A549 | ATCC ® CCL-185 | carcinoma | lung |
| H460 | ATCC ® HTB-177 | carcinoma; large cell lung | lung: pleural effusion |
| | | cancer | |
| H1299 | ATCC ® CRL-5803 | carcinoma; non-small cell | lung; derived from metastatic |
| | | lung cancer | site: lymph node |

The SK-ES-1, U-2 OS, and SAOS-2 cells were cultured in a growth medium that is ATCC-formulated McCoy's 5a Medium Modified, Catalog No. 30-2007. The PC-3, PANC-1, SK-Mel-5, A549, H460, and H1299 were cultured in a growth medium that is ATCC-formulated Dulbecco's Modified Eagle's Medium (DMEM), Catalog No. 30-2002. The cells were seeded on 96-well microtiter plates with 3500 cells per well and cultured in an incubator (Model 3120, Thermo Scientific, USA) with a constant temperature at 37° C. and 5% carbon dioxide (CO₂) gas for 24 hours.

The new tamibarotene molecular complexes were made into a series with concentration of 200 nM to 40 nM, 8 nM, 1.6 nM, 0.32 nM, and 0.064 nM. 100 μl of each solution was then added into each well to bring out the final concentration of the drug treatment at 100 nM, 20 nM, 4 nM, 0.8 nM, 0.16 nM, or 0.032 nM, respectively.

The treated cells in the 96 well plate was then further cultured at the same conditions for 72 hours and then subjected for a final cell viability measurement. The measurement used a homogeneous method to determine the number of metabolically active cells presented in each of treatments. A testing reagent for the measurement is a mixture solution of the CellTiter-Glo® 2.0 reagent (CTG, Promega Cat #G9243, USA) and growth medium in 1:1 ratio in this respect.

The 96-well plate with the new molecular complex treated cells was taken out from incubator after the 72 hours of incubation, and the treatment solution in each well was removed using a multichannel pipette, and 100 μl of the testing reagent were added into each well. The plate was then placed on a plate shaker for 15 min. The plate was covered with a piece of aluminum foil to protect the CTG luminescence during the shaking time.

Then 90 μl of the 100 μl testing reagent were transferred into an opaque-walled 96-well plate for the CTG luminescence reading on a plate reader (SynergyH4 Hybrid Reader, Biotek, USA). The luminescence intensity of each treatment was positively correlated with the number of cells survived during the treatments.

All the treatment data was normalized by comparing with the control samples that had only growth medium without any molecular complexes. The half maximal inhibitory concentration (IC₅₀) data was calculated using the nonlinear regression model and dose-responses (inhibition) curve built in the software. Each of treatments was performed in three replications each time and repeated three times for validation. The IC₅₀ average volume (Avg) and standard deviation (STDV) were calculated based on the three replications.

Tamibarotene molecular complexes have outperformed or possessed better potency comparing with the original parent compound (tamibarotene) in inhabiting cancer cell growth as suggested by the results in Table 2.

TABLE 2
The half maximal inhibitory concentration (IC₅₀) volume of the novel molecular complexes on
different cancer cells. IC₅₀ data were validated three times where each time a triplicate measurement
was conducted and their average (Avg) and standard deviation (STDV) is represented.

| Monolayer Cell Culture Assay | | | IC₅₀ Test(I) | | IC₅₀ Test (II) | | IC₅₀ Test (III) | |
|---|---|---|---|---|---|---|---|---|
| Cancer | | | Avg | | Avg | | Avg | |
| Type | Cell Line | Compounds | (μM) | STDV | (μM) | STDV | (μM) | STDV |
| Sarcoma | SK-ES-1 | TAM | >>100 | | 73.20 | 6.67 | 72.47 | 11.70 |
| | SK-ES-1 | TAM-gallic acid | 10.07 | 1.20 | 5.94 | 0.28 | 6.76 | 0.07 |
| Sarcoma | U-2 OS | TAM | >>100 | | 77.73 | 1.54 | 69.74 | 2.62 |
| | U-2 OS | TAM-decanoic acid | 85.05 | 3.80 | 65.91 | 2.43 | 65.84 | 6.74 |
| | U-2 OS | TAM-gallic acid | 16.37 | 0.31 | 13.03 | 0.51 | 10.55 | 0.46 |
| Sarcoma | SAOS | TAM | 55.38 | 4.47 | 46.47 | 0.29 | >100 | |
| | SAOS | TAM-biphenyl-4- | 43.53 | 5.72 | 37.06 | 5.25 | >100 | 32.68 |
| | | carboxylic acid | | | | | | |
| | SAOS | TAM-decanoic acid | 42.74 | 13.45 | 29.30 | 5.26 | 88.31 | 4.67 |
| | SAOS | TAM-gallic acid | 11.55 | 0.52 | 9.98 | 0.72 | 14.90 | 1.82 |
| Skin | SK-Mel-5 | TAM | >>100 | | >>100 | | >>100 | |
| | SK-Mel-5 | TAM-gallic acid | 47.46 | 14.63 | 64.21 | 6.89 | 88.49 | 8.10 |
| Prostate | PC-3 | TAM | >>100 | | >>100 | | >>100 | |
| | PC-3 | TAM-gallic acid | 80.95 | 0.24 | 90.28 | 16.41 | 107.52 | 7.60 |
| Lung | A549 | TAM | 59.90 | 2.94 | 60.64 | 3.19 | 57.80 | 4.79 |
| | A549 | TAM-biphenyl-4- | 49.30 | 1.74 | 41.27 | 2.52 | 46.34 | 2.33 |
| | | carboxylic acid | | | | | | |
| | A549 | TAM-decanoic acid | 53.64 | 6.37 | 59.08 | 0.80 | 48.98 | 7.78 |
| | A549 | TAM-diphenic acid | 21.73 | 5.13 | 38.87 | 0.55 | 26.64 | 4.40 |
| | A549 | TAM-gallic acid | 39.86 | 0.79 | 43.32 | 0.72 | 39.94 | 0.71 |
| | A549 | TAM-nicotinamide | 56.62 | 2.29 | 59.07 | 3.20 | 49.78 | 0.57 |
| Lung | H460 | TAM | 69.30 | 2.64 | 58.34 | 0.67 | 67.07 | 0.52 |
| | H460 | TAM-biphenyl-4- | 56.04 | 1.60 | 51.67 | 2.18 | 55.16 | 0.76 |
| | | carboxylic acid | | | | | | |
| | H460 | TAM-decanoic acid | 62.24 | 1.18 | 59.64 | 0.17 | 60.30 | 0.31 |
| | H460 | TAM-diphenic acid | 34.95 | 1.57 | 34.72 | 1.50 | 32.30 | 3.20 |
| | H460 | TAM-gallic acid | 42.00 | 0.88 | 41.86 | 0.80 | 41.27 | 1.84 |
| | H460 | TAM-nicotinamide | 56.57 | 0.17 | 51.06 | 3.71 | 50.34 | 1.63 |
| Lung | H1299 | TAM | 78.88 | 4.10 | 67.27 | 2.08 | 82.98 | 1.68 |
| | H1299 | TAM-biphenyl-4- | 65.51 | 0.77 | 59.78 | 1.29 | 65.31 | 2.75 |
| | | carboxylic acid | | | | | | |
| | H1299 | TAM-decanoic acid | 67.86 | 1.05 | 61.63 | 1.30 | 69.55 | 1.47 |
| | H1299 | TAM-diphenic acid | 40.75 | 1.27 | 41.39 | 1.35 | 39.86 | 2.06 |
| | H1299 | TAM-gallic acid | 11.22 | 0.60 | 24.75 | 0.84 | 11.67 | 0.60 |
| | H1299 | TAM-nicotinamide | 49.72 | 1.13 | 55.89 | 2.64 | 53.85 | 3.16 |

### Example 9: Tumor Sphere Formation Studies

Cancer stem cells (CSCs) are defined as a small subset of cells within a tumor with the ability to self-renew and often drive tumor progression and recurrence after chemotherapy treatment (Zhou et al. (2015) “A Reliable Parameter to Standardize the Scoring of Stem Cell Spheres,” PLOS One 10(5); e0127348). It is important to study the responses of cancer stem cells or the tumors treated with the new molecular complexes. We have assessed CSC growth using tumor sphere formation assay, which involves culturing cancer cells in low attachment plates in serum free media. Sphere size and diameter was measured using ImageJ software and statistical analysis was performed using Graph Pad Prism Software.

The stem cell culture medium is prepared for 500 ml (250 ml of Dulbecco's Modified Eagle Medium (DMEM) (ATCC® 30-2002) plus 250 ml of F-12K medium (ATCC® 30-2004)) using 20 ng/ml epidermal growth factor, 10 ng/ml basic fibroblast growth factor, 5 μg/ml insulin and 0.4% Bovine Serum Albumin. All preparation methods known to the person skilled in the art.

The cancer cell line used in this study was sarcoma cell line SK-ES-1. The cells were seeded in a density of 200 per well per 200 μl in a 96-well Ultra-Low Attachment plate (Corning, Cat #3474, USA), this plate is specially treated for stem cell growth or tumor culture. On the day 1 of seeding, 100 μl of cell solution was added into the plate. On the day 2, 100 μl of treatment solution, which is the stem cell culture medium mixed containing tamibarotene molecular complexes in this case, and were gently added into the treatment wells. The tamibarotene molecular complex concentrations prepared were 200 nM, 40 nM, 8 nM, 1.6 nM, 0.32 nM, and 0.064 nM. 100 μl of each solution was then added into each well to bring out the final concentration at 100 nM, 20 nM, 4 nM, 0.8 nM, 0.16 nM, or 0.032 nM, respectively.

On the day 6 of seeding, 10 μl of NucBlue Live Cell Stain ReadyProbes (Invitrogen Cat #R37605, USA) were added into each well for staining to visualize the live cells under the blue fluorescent light. The staining took place in an incubator for 2-3 hours at 37° C. Then the plate was brought to the Keyence imaging device (Keyence America, All-in-One Fluorescence Microscope BZ-X800, USA) for image scanning. The sphere images in each well were saved in “Tiff” file for data analysis. The ImageJ software (National Institutes of Health, USA) and its “particle analysis” function were used to identifying and measuring numbers and diameters of spheres by each treatment. The data only included the spheres with diameter over 50 μm.

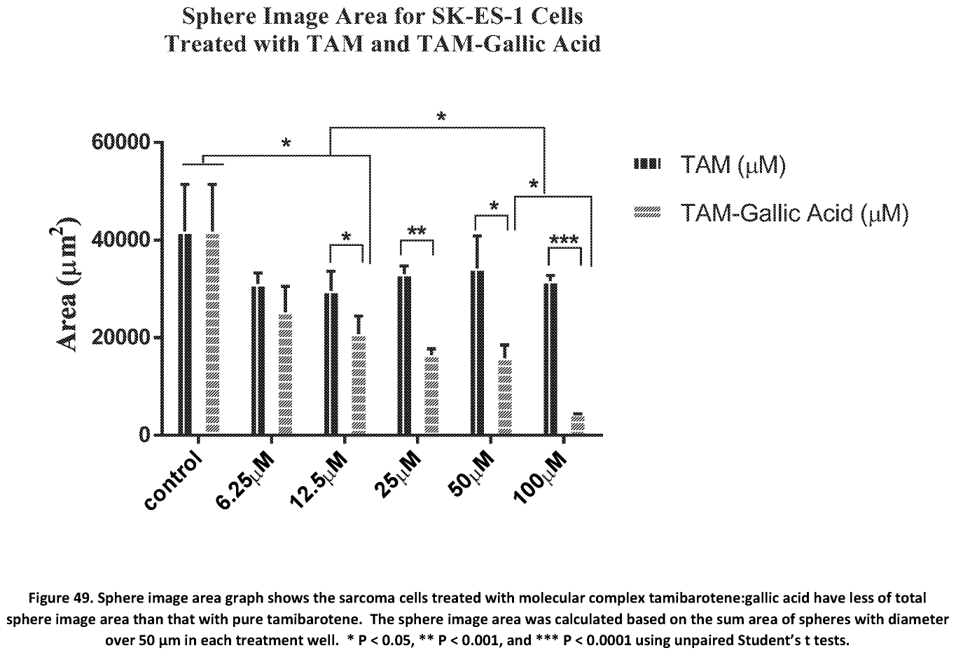

The sphere image area is calculated as a sum of image area of every individual sphere with diameter over 50 μm in each treatment well. GraphPad Prism (version 6) software was used for data analysis and graphing. Each of treatments was performed in three replications, and the average size and standard deviation (STDV) in error bar were expressed in FIG. 49. The treatment groups were compared with the control and each other using unpaired Student's t tests, and significance was declared at P<0.05 for the bars indicated by *, at P<0.001 by **, and P<0.0001 by ***.

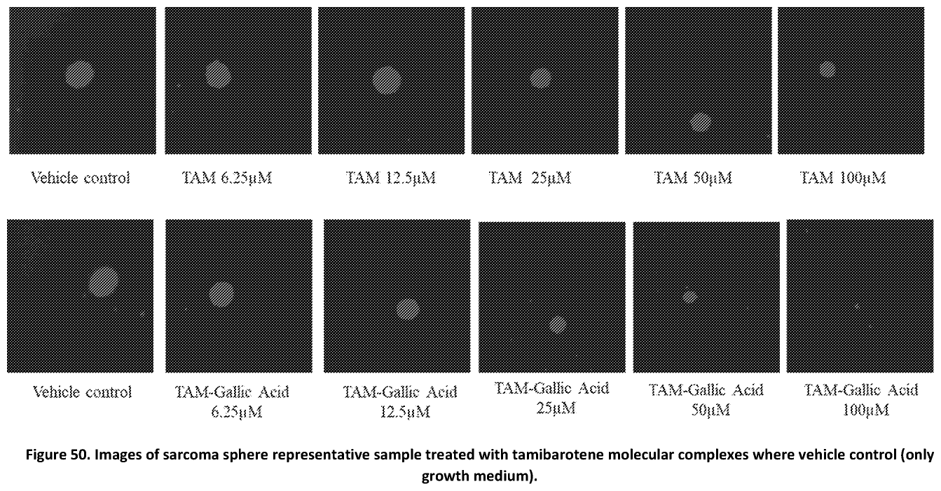

The sarcoma spheres were stained with NucBlue Live Cell Stain ReadyProbes (Invitrogen Cat #R37605) and imaged under 4× objective lens on Keyence imaging scanner (Keyence America, All-in-One Fluorescence Microscope BZ-X800). The images show that the sarcoma spheres treated with tamibarotene molecular complexes possess smaller sizes than that treated with tamibarotene parent molecule (FIG. 50) thus confirming the results of the calculated sphere image area. This finding suggests that the molecular complexes of tamibarotene were more potent for cancer treatment.

What is claimed:

1. A crystalline form of tamibarotene selected from the group consisting of: tamibarotene:adipic acid, tamibarotene:DL-aspartic acid, tamibarotene:acetylsalicylic acid, tamibarotene:biphenyl-4-carboxylic acid, tamibarotene:caffeic acid, tamibarotene:decanoic acid, tamibarotene:diphenic acid, tamibarotene:gallic acid, tamibarotene:fumaric acid, tamibarotene:ibuprofen, tamibarotene:maleic acid, tamibarotene:nicotinamide, tamibarotene:isonicotinamide, tamibarotene:citric acid, tamibarotene:nicotinic acid, tamibarotene:3,4-dihydroxybenzoic acid, tam ibarotene:glutaric acid, and tamibarotene:L-malic acid.

2. The crystalline form of claim 1, wherein the crystalline form is tamibarotene:adipic acid.

3. The crystalline form of claim 2, wherein the crystalline form is characterized by a powder X-ray diffraction pattern comprising one or more powder X-ray diffraction peaks selected from the group consisting of: about 10.5, 12.0, 14.5, 22.0, and 26.0° 2θ±0.2° 2θ.

4. The crystalline form of claim 1, wherein the crystalline form is tamibarotene:DL-aspartic acid.

5. The crystalline form of claim 4, wherein the crystalline form is characterized by a powder X-ray diffraction pattern comprising one or more powder X-ray diffraction peaks selected from the group consisting of: about 6.5, 10.0, 11.5, and 19.5° 2θ±0.2° 2θ.

6. The crystalline form of claim 1, wherein the crystalline form is tamibarotene:acetylsalicylic acid.

7. The crystalline form of claim 6, wherein the crystalline form is characterized by a powder X-ray diffraction pattern comprising one or more powder X-ray diffraction peaks selected from the group consisting of: about 8.0, 8.5, 15.5, 23.0, and 27.0° 2θ±0.2° 2θ.

8. The crystalline form of claim 1, wherein the crystalline form is tamibarotene:caffeic acid.

9. The crystalline form of claim 8, wherein the crystalline form is characterized by a powder X-ray diffraction pattern comprising one or more powder X-ray diffraction peaks selected from the group consisting of: about 3.5, 14.0, 16.0, 27.0, and 17.5° 2θ±0.2° 2θ.

10. The crystalline form of claim 1, wherein the crystalline form is tamibarotene:biphenyl-4-carboxylic acid.

11. The crystalline form of claim 10, wherein the crystalline form is characterized by a powder X-ray diffraction pattern comprising one or more powder X-ray diffraction peaks selected from the group consisting of: about 6.5, 8.0, 8.5, 11.0, 13.0, and 16.0° 2θ±0.2° 2θ.

12. The crystalline form of claim 1, wherein the crystalline form is tamibarotene:decanoic acid.

13. The crystalline form of claim 12, wherein the crystalline form is characterized by a powder X-ray diffraction pattern comprising one or more powder X-ray diffraction peaks selected from the group consisting of: about 4.0, 14.0, 15.0, 21.5, and 23.5° 2θ±0.2° 2θ.

14. The crystalline form of claim 1, wherein the crystalline form is tamibarotene:diphenic acid.

15. The crystalline form of claim 14, wherein the crystalline form is characterized by a powder X-ray diffraction pattern comprising one or more powder X-ray diffraction peaks selected from the group consisting of: about 8.0, 8.5, 13.0, 14.0, 14.5, and 16.0° 2θ±0.2° 2θ.

16. The crystalline form of claim 1, wherein the crystalline form is tamibarotene:gallic acid.

17. The crystalline form of claim 16, wherein the crystalline form is characterized by a powder X-ray diffraction pattern comprising one or more powder X-ray diffraction peaks selected from the group consisting of: about 3.5, 23.0, 28.5, and 29.5° 2θ±0.2° 2θ.

18. The crystalline form of claim 1, wherein the crystalline form is tamibarotene:fumaric acid.

19. The crystalline form of claim 18, wherein the crystalline form is characterized by a powder X-ray diffraction pattern comprising one or more powder X-ray diffraction peaks selected from the group consisting of: about 3.0, 6.5, 16.5, 18.0, and 21.5° 2θ±0.2° 2θ.

20. The crystalline form of claim 1, wherein the crystalline form is tamibarotene:ibuprofen.

21. The crystalline form of claim 20, wherein the crystalline form is characterized by a powder X-ray diffraction pattern comprising one or more powder X-ray diffraction peaks selected from the group consisting of: about 3.5, 7.0, 17.5, and 19.5° 2θ±0.2° 2θ.

22. The crystalline form of claim 1, wherein the crystalline form is tamibarotene:maleic acid.

23. The crystalline form of claim 22, wherein the crystalline form is characterized by a powder X-ray diffraction pattern comprising one or more powder X-ray diffraction peaks selected from the group consisting of: about 4.0, 6.0, 12.5, 14.5, and 17.5° 2θ±0.2° 2θ.

24. The crystalline form of claim 1, wherein the crystalline form is tamibarotene:nicotinamide.

25. The crystalline form of claim 24, wherein the crystalline form is characterized by a powder X-ray diffraction pattern comprising one or more powder X-ray diffraction peaks selected from the group consisting of: about 4.0, 7.5, 14.5, 15.5, and 19.5° 2θ±0.2° 2θ.

26. The crystalline form of claim 1, wherein the crystalline form is tamibarotene:isonicotinamide.

27. The crystalline form of claim 26, wherein the crystalline form is characterized by a powder X-ray diffraction pattern comprising one or more powder X-ray diffraction peaks selected from the group consisting of: about 8.0, 9.0, 21.5, 22.0, and 24.0° 2θ±0.2° 2θ.

28. The crystalline form of claim 1, wherein the crystalline form is tamibarotene:citric acid.

29. The crystalline form of claim 28, wherein the crystalline form is characterized by a powder X-ray diffraction pattern comprising one or more powder X-ray diffraction peaks selected from the group consisting of: about 6.5, 8.5, 12.5, 19.5, and 16.5° 2θ±0.2° 2θ.

30. The crystalline form of claim 1, wherein the crystalline form is tamibarotene:nicotinic acid.

31. The crystalline form of claim 30, wherein the crystalline form is characterized by a powder X-ray diffraction pattern comprising one or more powder X-ray diffraction peaks selected from the group consisting of: about 6.5, 8.5, 12.5, 16.5, 19.0, 19.5, and 25.0° 2θ±0.2° 2θ.

32. The crystalline form of claim 1, wherein the crystalline form is tamibarotene:3,4-dihydroxybenzoic acid.

33. The crystalline form of claim 32, wherein the crystalline form is characterized by a powder X-ray diffraction pattern comprising one or more powder X-ray diffraction peaks selected from the group consisting of: about 4.5, 9.5, 18.5, 23.5, and 24.5° 2θ±0.2° 2θ.

34. The crystalline form of claim 1, wherein the crystalline form is tamibarotene:glutaric acid.

35. The crystalline form of claim 34, wherein the crystalline form is characterized by a powder X-ray diffraction pattern comprising one or more powder X-ray diffraction peaks selected from the group consisting of: about 3.5, 7.0, 8.5, 14.5, and 21.5° 2θ±0.2° 2θ.

36. The crystalline form of claim 1, wherein the crystalline form is tamibarotene:L-malic acid.

37. The crystalline form of claim 36, wherein the crystalline form is characterized by a powder X-ray diffraction pattern comprising one or more powder X-ray diffraction peaks selected from the group consisting of: about 10.5, 14.0, 12.0, 19.5, and 24.5° 2θ±0.2° 2θ.

38. A composition comprising the crystalline form of any one of claims 1-37.

39. A pharmaceutical composition comprising the crystalline form of any one of claims 1-37 and at least one pharmaceutically acceptable excipient or cosmetically acceptable carrier.

40. The pharmaceutical composition of claim 39, where the pharmaceutical composition is suitable for any drug delivery route.

41. The pharmaceutical composition of claim 40, wherein the pharmaceutical composition is an oral dosage form, a topical dosage form, or an injectable dosage form.

42. The pharmaceutical composition of claim 39, wherein the pharmaceutical composition is a solid dosage form for reconstitution in at least one medium.

43. The pharmaceutical composition of claim 42, wherein the medium is an aqueous or oil based liquid.

44. The pharmaceutical composition of any one of claims 39-43, wherein the pharmaceutical composition is a unit dose.

45. A method of treating or preventing a disease for which tamibarotene is indicated, the method comprising the step of administering to a patient in need thereof, a therapeutically effective amount of a pharmaceutical composition of any one of claims 39-44.

46. The method of claim 44, wherein the disease is selected from the group consisting of: acute promyelocytic leukaemia (APL), Alzheimer's disease, multiple myeloma, Crohn's disease, systemic lupus erythematosus (SLE), cutaneous lupus erythematosus (CLE), drug-induced lupus, and neonatal lupus Wilms' tumor, rhabdomyosarcoma, lung, liver, breast, colon, rectal head and neck, brain, pancreatic, ovarian cancer, gestational trophoblastic neoplasm, Ewing's sarcoma, metastatic testicular tumors, gestational trophoblastic neoplasm, locally recurrent or locoregional solid tumors (sarcomas, carcinomas and adenocarcinomas), acute myeloid leukemia (AML), multiple myeloma, Shwachman-Diamond syndrome, prostate cancer, skin cancer, actinic keratosis, Bowen's disease, adjuvant cancer therapy, and neoadjuvant cancer therapy.

47. The method of claim 46, wherein the skin cancer is selected from the group consisting of: basal cell carcinoma (BCC), squamous cell carcinoma (SCC), and melanoma.

48. The method of claim 47, wherein the skin cancer is non-melanoma skin cancer.

49. The method of claim 46, wherein the disease is prostate cancer.

50. The method of claim 49, wherein prostate cancer is selected from the group consisting of: acinar adenocarcinoma, ductal adenocarcinoma, transitional cell (or urothelial) cancer, squamous cell cancer, small cell prostate cancer, carcinoid, and sarcoma.

51. The method of claim 45, wherein the disease is acute promyelocytic leukaemia (APL).

52. The method of claim 38, wherein the crystalline forms have improved (IC₅₀) compared with that of tamibarotene alone.

53. The method of claim 52, wherein the crystalline forms of tamibarotene:biphenyl-4-carboxylic acid, tamibarotene:decanoic acid, tamibarotene:diphenic acid, tamibarotene:gallic acid, tamibarotene:nicotinamide, and tamibarotene:L-malic acid have improved (IC₅₀) compared with that of tamibarotene alone.

54. The method of claim 53, wherein the crystalline form is tamibarotene:gallic acid.

55. A method of eliminating cancer stem cells using the crystalline form of any one of claims 1-37.

56. The method of claim 55, wherein the crystalline form is tamibarotene:gallic acid.

57. The method of claim 55, wherein the method comprises the step of administering to a patient in need thereof a therapeutically effective amount of a pharmaceutical composition of any one of claims 39-43.

58. A method of eliminating tumoroids using the crystalline form of any one of claims 1-37.

59. The method of claim 58, wherein the crystalline form is tamibarotene:gallic acid.

60. The method of claim 57, wherein the method comprises the step of administering to a patient in need thereof a therapeutically effective amount of a pharmaceutical composition of any one of claims 39-43.

61. The method of any one of claims 45-51, wherein the pharmaceutical composition is administered topically or via intratumoral injection.

62. A method of making the crystalline form of any one of claims 1-37, comprising the steps of: combining tamibarotene and a former selected from the group consisting of: adipic acid, DL-aspartic acid, acetylsalicylic acid, biphenyl-4-carboxylic acid, caffeic acid, decanoic acid, diphenic acid, gallic acid, fumaric acid, ibuprofen, maleic acid, nicotinamide, isonicotinamide, citric acid, nicotinic acid, 3,4-dihydroxybenzoic acid, glutaric acid, and L-malic acid; and forming crystals of the tamibarotene and the former.

63. The method of claim 62, wherein the method comprises the step of combining the tamibarotene and the former with a solvent.

64. The method of claim 63, wherein the solvent is selected from the group consisting of: acetone, ethanol, methanol, ethylacetate (EtOAc), isopropanol (IP A), isopropylacetate (IP Ac), diethoxymethane (DEM), toluene, BuOAc, N-methylpyrrolidone (NMP), and a heptane.
